SEQUENCE LISTING (10 sequences)

SEQ ID NO: 1 - protein, 5 aa, Artificial Sequence
  Heavy chain CDR1
SYDIN

SEQ ID NO: 2 - protein, 17 aa, Artificial Sequence
  Heavy chain CDR2
WIYPGDGSIKYNEKFKG

SEQ ID NO: 3 - protein, 13 aa, Artificial Sequence
  Heavy chain CDR3
RGEYGNYEGAMDY

SEQ ID NO: 4 - protein, 15 aa, Artificial Sequence
  Light chain CDR1
KASQSVDYDGHSYMN

SEQ ID NO: 5 - protein, 7 aa, Artificial Sequence
  Light chain CDR2
AASNLES

SEQ ID NO: 6 - protein, 9 aa, Artificial Sequence
  Light chain CDR3
QQSDENPLT

SEQ ID NO: 7 - protein, 122 aa, Artificial Sequence
  Heavy chain variable region (VH)
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 8 - protein, 112 aa, Artificial Sequence
  Light chain variable region (VL)
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 9 - protein, 448 aa, Artificial Sequence
  Heavy chain
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 218 aa, Artificial Sequence
  Light chain
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0378912 A1
(43) Pub. Date: Dec. 1, 2022

(54) CRIZANLIZUMAB CONTAINING ANTIBODY FORMULATIONS

(71) Applicant: NOVARTIS AG, Basel (CH)

(72) Inventors: Fabian BICKEL, Basel (CH); Lina BOADO, Basel (CH); Dirk CHELIUS, Basel (CH); Francois GRIAUD, Basel (CH); Caroline HILBERT, Village-Neuf (FR); Frieder KROENER, Basel (CH); Juergen SIGG, Loerrach (DE); Rajsekhar PAUL, Basel (CH); Maja ANKO, Menges (SK); Aljosa JELENKO, Menges (SK)

(21) Appl. No.: 17/771,353

(22) PCT Filed: Oct. 29, 2020
(86) PCT No.: PCT/US2020/057868

§ 371 Date: Apr. 22, 2022

Related U.S. Application Data
(60) Provisional application No. 62/927,716, filed on Oct. 30, 2019.
(60) Provisional application No. 62/927,720, filed on Oct. 30, 2019.
(60) Provisional application No. 62/933,692, filed on Nov. 11, 2019.
(60) Provisional application No. 62/936,269, filed on Nov. 15, 2019.

(51) Int. Cl. A61K 39/395; A61K 47/02; A61K 47/26; A61K 9/19; C07K 16/28

(52) U.S. Cl. CPC A61K 39/39591; A61K 47/02; A61K 47/26; A61K 9/19; C07K 16/2854; C07K 2317/565; C07K 2317/94; A61K 9/0019

(57) ABSTRACT

The present invention relates to novel pharmaceutical formulations of an antibody against human P-selectin, especially SEG101, or an antibody having at most 3 amino acid difference from crizanlizumab, and processes for the preparation thereof and uses of the formulations.

### FIELD OF INVENTION

The present invention relates to novel pharmaceutical formulations of an antibody against human P-selectin, especially SEG101, or an antibody having at most 3 amino acid difference from crizanlizumab, and processes for the preparation thereof and uses of the formulations.

### BACKGROUND

P-selectin contributes to many inflammatory and thrombotic diseases. Accordingly, therapeutics targeting P-selectin, such as antibodies against human P-selectin as disclosed in WO 2008/069999, especially SEG101 (a.k.a. crizanlizumab and SelG1), can be used as a means of treating inflammatory and thrombotic diseases.

Formulated antibodies may lose biological activity resulting from chemical and physical instabilities during the storage. Degradation of the antibody or even the excipients, formation of charge variants and isomerization reactions are among the common factors leading to safety concerns and decreased potency and efficacy of the formulation over time.

Conveniently, liquid pharmaceutical formulations of protein therapeutics, e.g. antibodies, should be long-term stable and contain a safe and effective amount of the rapeutics. In addition to the challenges relating to the physical and chemical stability, such as formation of aggregates and difficulty with manufacture, storage, and delivery, problems with liquid formulations of protein therapeutics are the degradation and the formation of charge variants, which can negatively influence the activity and functionality of the protein of interest, besides causing safety issues. Thus, there is a need for development of formulations capable of maintaining the protein therapeutics stable over a long time, minimizing the degradation rate and formation of other molecular species such as charge variants and/or isoforms.

### BRIEF SUMMARY OF THE DISCLOSURE

It is an object of the present invention to provide an anti-P-selectin antibody formulation, in particular for crizanlizumab or an antibody having at most 3 amino acid difference from crizanlizumab, which is stable upon storage and delivery. It is a further object to provide a stable liquid antibody formulation which is suitable for intravenous (i.v.) administration.

In one aspect, the present invention provides a pharmaceutical composition comprising crizanlizumab that has light chain and heavy chain amino acid sequences of SEQ ID NO: 10 and SEQ ID NO: 9 respectively, and a variant of crizanlizumab (iso-crizanlizumab), in which the amino acid aspartic acid at position 32 of SEQ ID NO: 10 is changed to isoaspartic acid. Suitably and preferably, the pharmaceutical composition further comprises a buffering system, resulting in pH value from 5.5 to 7.5, preferably from 5.7 to 7.0, preferably from 5.7 to 6.3 for the pharmaceutical composition.

A pharmaceutical composition must be suitable to be administered to a human subject. Apart from the effects elicited from the active ingredient contained, the pharmaceutical composition should be suitable for use in contact with the tissues of human beings without excessive toxicity, irritation, allergic response, or other problem or complication, commensurate with a reasonable benefit/risk ratio.

In one aspect, the invention relates to novel pharmaceutical compositions comprising an antibody to human P-selectin, preferably crizanlizumab, or an antibody having at most 3 amino acid difference from crizanlizumab, wherein the pH value is from 5.5 to 7.5, 5.7 to 7.0, preferably from 5.7 to 6.3.

### BRIEF DESCRIPTION OF THE DRAWINGS

FIG. 1. Impact of formulation pH on CZE Main Peak.

FIG. 2. Impact of formulation pH on CZE basic peaks.

FIG. 3. Impact of formulation pH on CZE acidic peaks.

FIG. 4. Purity of screened formulations by SEC.

FIG. 5. pH dependent isomerization of crizanlizumab

### DEFINITIONS

In order that the present disclosure may be more readily understood, certain terms are first defined. Additional definitions are set forth throughout the detailed description.

As used herein, the term “a”, “an”, “the” and similar terms used in the context of the present disclosure (especially in the context of the claims) are to be construed to cover both the singular and plural unless otherwise indicated herein or clearly contradicted by the context. As such, the terms “a” (or “an”), “one or more”, and “at least one” can be used interchangeably herein.

“And/or” means that each one or both or all of the components or features of a list are possible variants, especially two or more thereof in an alternative or cumulative way.

The term “about” in relation to a numerical value X means, for example, X±10%, X 5%, X±3%, including all the values within this range.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” or “subject” are taken to mean humans. Except when noted, the terms “patient” or “subject” are used herein interchangeably.

As used herein, a subject is “in need of” a treatment if such subject would benefit biologically, medically or in quality of life from such treatment.

The term “treatment” includes: (1) preventing or delaying the appearance of clinical symptoms of the state, disorder or condition developing in an animal, particularly a mammal and especially a human that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be an effective treatment.

### DETAILED DESCRIPTION

Liquid formulations of protein therapeutics should preserve intact the biologic activity of the protein therapeutics and protect the functional groups of the protein therapeutics from degradation during manufacturing and shelf life. Degradation pathways for proteins can involve chemical instability (e.g. deamidation, oxidation, clipping, isomerization etc.) or physical instability (e.g. formation of aggregates). Different degradation products of an antibody can be detected as charge variants by methods well known in the art e.g. capillary isoelectric focusing (cIEF), ion exchange chromatography or capillary zone electrophoresis (see e.g. doi: 10.1016/j.jchromb.2017.02.017 and doi: 10.4161/mabs.2.6.13333). Common charge variants are sialic acid, deamidation, C-terminal lysine, N-terminal glutamic acid, unprocessed leader sequence, isomerization of aspartic acid and formation of succinimide (Yi Du, et al., 2012. DOI: 10.4161/mabs.21328).

The microheterogeneity of crizanlizumab is related to modifications or chemical degradations, which result in large amount of differently modified variants. A major variant for crizanlizumab is formed via isomerization of aspartic acid at position 32 of the light chain (in the CDR) to isoaspartic acid (giving rise to a variant herein referred to as “iso-crizanlizumab”) through the step of formation of a cyclic imide intermediate (succinimide), which can be hydrolysed into aspartic acid and isoaspartic acid with a molar ratio of approximately 1:3. It has been however surprisingly found that the iso-crizanlizumab shows biological activity substantially equal to that of crizanlizumab although this modification is in the CDR. Furthermore, the corresponding succinimide intermediate is capable of being hydrolysed to aspartic acid and isoaspartic acid (giving rise to crizanlizumab and iso-crizanlizumab, respectively) under physiological conditions, meaning that the succinimide variant can be converted to biologically active forms after being administered to patients. In accordance with this finding, the pharmaceutical composition of the present invention, although containing crizanlizumab and a large amount of different variants, retains biological activity of crizanlizumab and is stable for a long period of time. In addition, the presence of iso-crizanlizumab and succinimide of crizanlizumab does not present immunogenecity issue. The term “immmunogenicity” refers to the generation of host antibodies that are capable of binding to crizanlizumab after administration of the pharmaceutical composition of the invention into a human subject, such as a healthy volunteer or a patient in need thereof. Less than 2% of human subjects, among all who received the pharmaceutical composition of the invention, have developed anti-crizanlizumab antibodies. On one hand under the physiological condition, suitably one day after drug administration, less than 5%, suitably less than 2%, suitably less than 1%, suitably less than 0.5% of the total crizanlizumab and all its variants in a patient is succinimide of crizanlizumab. Furthermore it is surprising to find that iso-crizanlizumab is as low immunogenic, substantially the same as crizanlizumab. The term “substantially the same” as used in this context means that immunogenecity of iso-crizanlizumab is not more than 5 fold, not more than 3 fold, suitably not more than 2 fold, suitably not more than 1.5 fold higher than that of crizanlizumab, when tested under the same conditions. Alternatively the term “substantially the same” as used in this context means that immunogenecity of iso-crizanlizumab is not less than 20%, suitably not less than 40%, suitably not less than 70% than that of crizanlizumab, when tested under the same conditions.

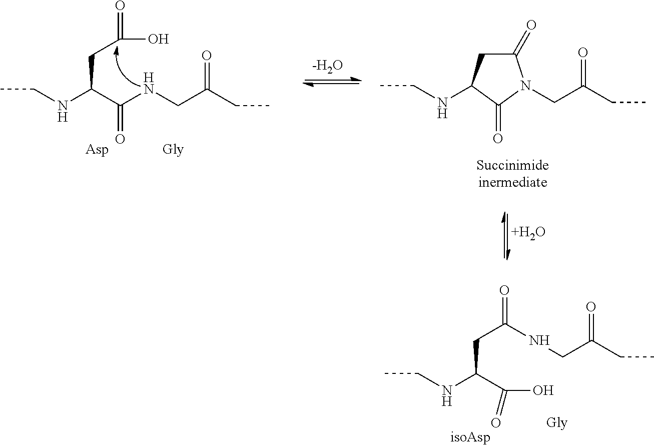

Thus in one aspect, the present invention provides a pharmaceutical composition comprising crizanlizumab that has light chain and heavy chain amino acid sequences of SEQ ID NO: 10 and SEQ ID NO: 9 respectively, and a variant of crizanlizumab, in which amino acid aspartic acid at position 32 of SEQ ID NO: 10 is changed to isoaspartic acid (iso-crizanlizumab).

The term “iso-crizanlizumab” consists of two species homo-iso-crizanlizumab and hetero-iso-crizanlizumab. The term homo-iso-crizanlizumab refers to the antibody in which aspartic acid residues at position 32 of both of the light chains are replaced by (i.e. isomerised to) isoaspartic acid. The term hetero-iso-crizanlizumab refers to the antibody in which aspartic acid residue at position 32 of only one of the light chains is replaced by isoaspartic acid.

As far as a single light chain is concerned, a light chain having aspartic acid at position 32 is named LC_{D}. A light chain having iso-apartic acid at position 32 is named LC_{isoD}. A light chain having succinimide at position 32 is named LC_{succi}.

In one aspect, the present invention provides a pharmaceutical composition comprising an antibody having at most 3, preferably 2, more preferably only one amino acid difference from crizanlizumab having light chain and heavy chain amino acid sequences in SEQ ID NO: 10 and SEQ ID NO: 9 respectively, and a variant of said antibody, in which aspartic acid at position 32 of SEQ ID NO: 10 is changed to isoaspartic acid.

Regarding an antibody having at most 3, preferably 2, more preferably only one amino acid difference from crizanlizumab, the difference can be a mismatch, i.e. replacement of a residue with a different residue. The difference can also be an insertion or deletion of an amino acid residue in the sequence of crizanlizumab. The difference can be anywhere in the sequence of the light and/or heavy chains, for example in different domains of the antibody, e.g. in CH1, CH2, CH3, VH, CL, VL, hinge region, F_{c} and F_{ab}. Preferably, said differences do not interfere with fundamental properties of the antibody such as its ability to bind its antigen, i.e. human P-selectin. Preferably, the differences are not in positions that are critical for the function of the antibody, e.g. CDRs. In particular, in the context of the pharmaceutical composition of the invention, the difference is not at position 32 of SEQ ID NO:10.

In one embodiment, the pharmaceutical composition further comprises a variant being succinimide (i.e. succinimide isoform of aspartic acid) at position 32 of SEQ ID NO:10 (“succinimide of crizanlizumab”). Succinimide of crizanlizumab is typically an antibody having in position 32 of at least one of the light chains succinimide and in position 32 of the other light chain either aspartic acid or isoaspartic acid, or in very acidic condition (e.g. pH around 5), both aspartic acids could be replaced with succinimide. The antibody comprising one light chain of succinimide and the other light chain aspartic acid in position 32 is named “D-succinimide of crizanlizumab”. The antibody comprising one light chain having succinimide and the other light chain having iso-aspartic acid in position 32 is named “isoD-succinimide of crizanlizumab”.

In one embodiment, the pharmaceutical composition comprises at least 20%, suitably at least 24%, at least 30%, suitably at least 35%, suitably at least 40% crizanlizumab (i.e. main peak) of the total charge variants in the pharmaceutical composition. Typically and preferably, the charge variants can be analysed by capillary zone electrophoresis (CZE). The area under curve (AUC) will be generally used to determine the % of each peak against the total AUC. For the sake of clarity, the peak comprising crizanlizumab will be referred as the “main peak” throughout this application, even in the rare situations, in which the peak comprising crizanlizumab does not have the largest AUC among all the peaks.

The term “charge variant” as used in this application refers to all peaks that can be identified by CZE, including the peak of crizanlizumab. The term “basic variant” as used in this application refers to the peaks corresponding to the lower time values compared to crizanlizumab in the CZE diagram. The term “acidic variant” as used in the application refers to the peaks corresponding to the higher time values compared to the peak of crizanlizumab in the CZE diagram. CZE can be performed, for example, as described in the Example 4.

There are 2 major acidic variant peaks. The one with lower time value comprises hetero-iso-crizanlizumab and the one with higher time value comprises homo-iso-crizanlizumab. By estimation in total iso-crizanlizumab accounts for about 50% to about 75% of the acidic variants.

There are a few basic variant peaks, among which one with lower time value comprises D-succinimide of crizanlizumab, and the other one with higher time value comprises isoD-succinimide of crizanlizumab. By estimation in total succinimide of crizanlizumab accounts for about 25% to about 50% of the basic variants.

In one embodiment, the pharmaceutical composition, when subjected to CZE, displays a main peak, the AUC of which is at least 20%, suitably at least 24%, at least 30%, suitably at least 35%, suitably at least 40% of the total AUC.

In one embodiment, the pharmaceutical composition comprises at most 70%, suitable at most 60%, suitably at most 50%, suitably at most 45% crizanlizumab of the total charge variants.

In one embodiment, the pharmaceutical composition, when subjected to CZE, displays a main peak, the AUC of which is at most 70%, suitable at most 60%, suitably at most 50%, suitably at most 45% of the total AUC.

In one embodiment, the pharmaceutical composition comprises from about 20% to about 50%, suitably from about 35% to about 45%, suitably from about 37% to about 42% crizanlizumab of the total charge variants.

In one embodiment, the pharmaceutical composition comprises from about 20% to about 50%, suitably from about 35% to about 45%, suitably from about 37% to about 42% main peak of the total charge variants.

In one embodiment, the pharmaceutical composition comprises at least 20%, at least 30%, iso-crizanlizumab of the total charge variants.

In one embodiment, the pharmaceutical composition comprises at least 30%, suitably at least 40% acidic variants of the total charge variants.

In one embodiment, the pharmaceutical composition comprises at most 40%, suitably at most 35% iso-crizanlizumab of the total charge variants.

In one embodiment, the pharmaceutical composition comprises at most 56%, suitably at most 45% acidic variants of the total charge variants.

In one embodiment, the pharmaceutical composition comprises from about 20% to about 40%, suitably from about 25% to about 35% iso-crizanlizumab of the total charge variants.

In one embodiment, the pharmaceutical composition comprises from about 30% to about 50%, suitably from about 35% to about 45%, suitably from about 37% to about 42% acidic variants of the total charge variants.

In one embodiment, the pharmaceutical composition comprises at least 5%, suitably at least 10% succinimide of crizanlizumab of the total charge variants.

In one embodiment, the pharmaceutical composition comprises suitably at least 10% basic, at least 15% basic variants of the total charge variants.

In one embodiment, the pharmaceutical composition comprises at most 15%, suitably at most 20% succinimide of crizanlizumab of the total charge variants.

In one embodiment, the pharmaceutical composition comprises at most 20%, suitably at most 25%, suitably at most 35% of basic variants of the total charge variants.

In one embodiment, the pharmaceutical composition comprises from about 5% to about 20%, suitably from about 10% to 15% of succinimide of crizanlizumab of the total charge variants.

In one embodiment, the pharmaceutical composition comprises from about 10% to about 35%, suitably from about 15% to 25% of basic variants of the total charge variants.

In one embodiment, the pharmaceutical composition comprises at least about 55%, at least about 60% of crizanlizumab plus iso-crizanlizumab of the total charge variants.

In one embodiment, the pharmaceutical composition comprises from about 55% to about 85%, from about 60% to about 80%, from about 65% to about 75% of crizanlizumab plus iso-crizanlizumab of the total charge variants.

In one embodiment, the present invention provides a pharmaceutical composition comprising crizanlizumab that has light chain and heavy chain amino acid sequences of SEQ ID NO: 10 and SEQ ID NO: 9 respectively, and a variant of crizanlizumab in which amino acid aspartic acid at position 32 of SEQ ID NO: 10 is changed to isoaspartic acid (iso-crizanlizumab), and a variant being succinimide at position 32 of SEQ ID NO:10 (succinimide of crizanlizumab).

Using capillary zone electrophoresis (CZE) a pH-dependent dynamic behavior of the charge variant distribution was identified. During incubation at pH below 6.3, succinimide accumulation occurred.

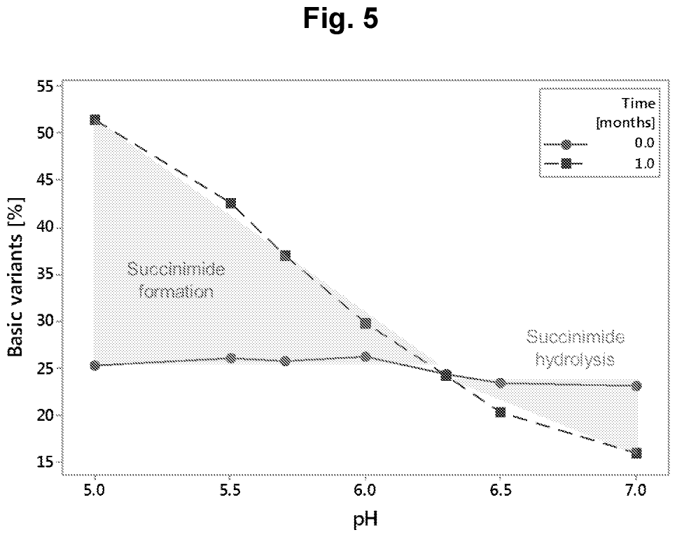

The lower the incubation pH is, the more succinimide accumulation. At pH values above 6.3 more isoaspartic acid was formed, while the initial amounts of succinimide were decreased to levels even lower than observed in starting material (FIG. 5).

Chemical modifications in antibody's complementarity-determining regions (CDR) can affect the binding activity to the target molecule. The influence of succinimide as isomerization variant in the CDR on the binding activity has been already reported (Yan B et al., 2009, doi: 10.1002/jps.21655; Cacia J et al., 1996, doi: 10.1021/bi951526c; Valliere-Douglass J et al., 2008, doi: 10.1016/j.chroma.2008.10.078; Ouellette D et al., 2013, doi: 10.4161/mabs.24458). In addition, isoaspartic acid as isomerization variant has also been shown to cause a decreased binding activity, if present in the CDR (Cacia J et al., 1996, doi: 10.1021/bi951526c; Harris RJ et al., 2001, DOI: 10.1016/s0378-4347(00)00548-x; Rehder DS et al., 2008, doi: 10.1021/bi7018223). While it is found that the potency of the basic fractions (containing succinimide) was reduced compared to the potency of the main peak (containing aspartic acid, crizanlizumab), it is surprising to find that the acidic variants containing iso-crizanlizumab retain biological activity substantially equal to that of crizanlizumab.

Thus in one aspect, the present invention provides an isolated variant of crizanlizumab comprising light chain and heavy chain amino acid sequences in SEQ ID NO: 10 and SEQ ID NO: 9 respectively, wherein the amino acid aspartic acid at position 32 of SEQ ID NO: 10 is replaced with isoaspartic acid either in one of the light chains or in both of the light chains of an antibody (iso-crizanlizumab). In one embodiment, the replacement is through the formation of a succinimide intermediate. In one aspect, the present invention provides a pharmaceutical composition comprising crizanlizumab, iso-crizanlizumab and succinimide of crizanlizumab, wherein the binding affinity of iso-crizanlizumab to human P-selectin is substantially equal with the binding affinity of crizanlizumab to human P-selectin. The binding affinity of crizanlizumab or iso-crizanlizumab to P-selectin can be determined by routine methodologies, for example by ELISA (e.g. as in the Example 2.1). The term “substantially equal” in this context is understood that the binding affinity of crizanlizumab and iso-crizanlizumab is not different by more than 2 fold, suitably not more than 1.5 fold, suitably not more than 1.3 fold, suitable not more than 1.2 fold. Suitably the binding affinity of iso-crizanlizumab is within 80% to 125% of the binding affinity of crizanlizumab.

In one embodiment, the biological activity of iso-crizanlizumab is substantially equal with the biological activity of crizanlizumab. The term “biological activity of crizanlizumab” refers to the ability of crizanlizumab to inhibit the interaction of human P-selectin with its ligand PSGL-1 (P-selectin glycoprotein ligand-1), typically to inhibit the interaction of cells expressing human P-selectin with PSGL-1. The biological activity can be determined by routine methodologies.

Suitably it is determined by measuring the fluorescence signal from the microtiter plates whose wells are first coated with PSGL-1 and then incubated with mammalian cells expressing human P-selection on their surface, after the cells are fluorescently labelled and incubated with SEG101 comprised in the pharmaceutical composition of the present invention. A suitable way of measuring the biological activity of SEG101 or its variants is demonstrated in Example 2.1. The term “substantially equal” in this context is understood that the biological activity of crizanlizumab and iso-crizanlizumab is not different by more than 2 fold, suitably not more than 1.5 fold, suitably not more than 1.3 fold, suitable not more than 1.2 fold. Suitably the biological activity iso-crizanlizumab is within 80% to 125% of the biological activity of crizanlizumab.

In another aspect, the present invention provides an isolated variant of crizanlizumab (succinimide of crizanlizumab) comprising light chain and heavy chain amino acid sequences in SEQ ID NO: 10 and SEQ ID NO: 9 respectively, wherein the amino acid aspartic acid at position 32 of SEQ ID NO: 10 is replaced with succinimide in at least one of the light chains, whereas the corresponding position at the other light chain is aspartic acid, isoaspartic acid or succinimide.

In one aspect, the present invention provides a pharmaceutical composition comprising crizanlizumab, iso-crizanlizumab and succinimide of crizanlizumab, wherein succinimide of crizanlizumab is capable of being hydrolysed to iso-crizanlizumab and crizanlizumab. In one embodiment, succinimide of crizanlizumab is capable of being hydrolysed to iso-crizanlizumab and crizanlizumab at pH 7.4±0.4. In one embodiment, succinimide of crizanlizumab is capable of being hydrolysed to iso-crizanlizumab and crizanlizumab at pH 7.4±0.4 at room temperature, typically at about 25° C. In one embodiment, succinimide of crizanlizumab is capable of being hydrolysed to iso-crizanlizumab and crizanlizumab under physiological conditions. The term “physiological condition” is understood as pH 7.4±0.4 and temperature between about 36.0 to about 40.0, preferably between 36.5 to 38.0, preferably about 36.5 to 37.5° C. In one embodiment, about 50% succinimide of crizanlizumab is hydrolysed to iso-crizanlizumab and crizanlizumab under physiological conditions within about 2 to about 5 hours, suitably within about 3 to about 5 hours, suitably with about 3 to about 4 hours. In one embodiment, succinimide of crizanlizumab is hydrolysed to iso-crizanlizumab and crizanlizumab after injection (e.g. intravenously) to a subject.

In one embodiment, in the pharmaceutical composition crizanlizumab is the main species as determined by CZE. Main species is understood as having the largest AUC.

In one embodiment, in the pharmaceutical composition iso-crizanlizumab is the main species as determined by CZE.

In one embodiment, the pharmaceutical composition comprises substantially equal amount of crizanlizumab and iso-crizanlizumab. The term “substantially equal” as used in this context means that the amount of iso-crizanlizumab is within 80% to 125% of the amount of crizanlizumab, suitably within 90% to 110% of crizanlizumab.

In one embodiment, the pharmaceutical composition of the present invention is kept at about 2° C. to about 8° C., suitably about 5° C. Suitably the pharmaceutical composition is kept in refrigerator. In one embodiment, the pharmaceutical composition of the present invention has a temperature between about 2° C. to about 8° C., suitably between about 4° C. to about 6° C., suitably 5° C. It is found out that the isomerization of aspartic acid at position 32 takes place at a much higher speed with increased temperature (as shown, e.g., in table 6). Under such temperature conditions, the decrease of crizanlizumab, preferably as determined as the AUC of the main peak in the CZE test, is not more than 25%, not more than 20%, not more than 15%, suitably not more than 10%, suitably not more than 5% over a period of at least 12 months, suitably for a period of 18 months, suitably for a period of 24 month, as compared to the starting material, which is at the time when the pharmaceutical composition is prepared. Under such temperature conditions, the amount of the total basic variants does not change more than 15%, not more than 10%, not more than 5%, suitably not more than 3%, suitable not more than 2% as compared to the starting material over a period of at least 12 months, suitably for a period of 18 months, suitably for a period of 24 month. Under such temperature conditions, the amount of the total acidic variants does not increase more than 25%, not more than 15%, suitably not more than 10%, suitably not more than 5% over a period of at least 12 months, suitably for a period of 18 months, suitably for a period of 24 month, as compared to the starting material. The above changes can be further minimized if the pharmaceutical composition has a proper pH range. For example at the pH between about 5.5 to about 7.5, suitably at the pH between about 5.5 to about 7, suitably at the pH between about 6 to about 7, suitably about 6±0.3. Suitably the pharmaceutical formulation is kept at pH about 6.

In one embodiment, the present invention provides a pharmaceutical composition comprising at least 24%, at least 30%, at least 35% main peak and at most 56%, at most 50%, at most 45% acidic variants of the total charge variants, suitably at the time of 18 months or 24 months shelf life, suitably the pharmaceutical composition is kept at about 2 to about 8° C., suitably about 5° C. In one embodiment, the present invention provides a pharmaceutical composition comprising at least 24% main peak and at most 56% acid variants of the total charge variants, suitably at the time of 18 months or 24 months shelf life, suitably the pharmaceutical composition is kept at about 2 to about 8° C., suitably about 5° C.

In one embodiment, the present invention provides a pharmaceutical composition comprising at least 24%, at least 30%, at least 35% main peak, at most 56%, at most 50%, at most 45% acidic variants, at most 35%, at most 30%, at most 25% of basic variants of the total charge variants, suitably at the time of 18 months or 24 months shelf life, suitably the pharmaceutical composition is kept at about 2 to about 8° C., suitably about 5° C. In one embodiment, the present invention provides a pharmaceutical composition comprising at least 24% main peak, at most 56% acidic variants, and at most 35% basic variants of the total charge variants, suitably at the time of 18 months or 24 months shelf life, suitably the pharmaceutical composition is kept at about 2 to about 8° C., suitably about 5° C.

Mass spectrometry (MS) analysis is used to identify chemical modifications, including LC_{D}, LC_{isoD} and LC_{succi}. In one aspect, the present invention provides a pharmaceutical composition comprising at least 50% LC_{D} of the total amount of LC_{D}, LC_{isoD} and LC_{succi} as determined by MS. In one embodiment, the present invention provides a pharmaceutical composition comprising from about 60% to about 85% of LC_{D} of the total amount of LC_{D}, LC_{isoD} and LC_{succi}. In one embodiment, the present invention provides a pharmaceutical composition comprising from about 70% to about 85% of LC_{D} of the total amount of LC_{D}, LC_{isoD} and LC_{succi} within the first 3 months of the shelf life. In one embodiment, the present invention provides a pharmaceutical composition comprising from about 60% to about 70% of LC_{D} of the total amount of LC_{D}, LC_{isoD} and LC_{succi} after the 12 months of the shelf life, suitably 18 months of shelf life, suitably 24 months of shelf life. In one embodiment, the present invention provides a pharmaceutical composition comprising LC_{D}, wherein the percentage decrease of LC_{D} over the total amount of LC_{D}, LC_{isoD} and LC_{succi} is not more than about 30%, not more than 20%, suitably not more than 15% from the beginning of shelf life to the end of the shelf life, wherein shelf life is 12 months, suitably 18 months, suitably 24 months. In one embodiment, the present invention provides a pharmaceutical composition comprising LC_{D}, wherein the percentage decrease of LC_{D} over the total amount of LC_{D}, LC_{isoD} and LC_{succi} is not more than about 20% for a period of about at least 18 months. Suitably the pharmaceutical composition is kept at about 2 to about 8° C., suitably about 5° C. The above changes can be further minimized if the pharmaceutical composition has a proper pH range. For example at the pH between about 5.5 to about 7.5, suitably at the pH between about 5.5 to about 7, suitably at the pH between about 6 to about 7, suitably about 6±0.3. Suitably the pharmaceutical formulation is kept at pH about 6.

In one embodiment, the present invention provides a pharmaceutical composition comprising at least 10% LC_{isoD} of the total amount of LC_{D}, LC_{isoD} and LC_{succi} as determined by MS. In one embodiment, the present invention provides a pharmaceutical composition comprising at most 30% LC_{isoD} of the total amount of LC_{D}, LC_{isoD} and LC_{succi} as determined by MS. In one embodiment, the present invention provides a pharmaceutical composition comprising from about 10% to about 30% LC_{isoD}, suitably from about 15% to about 25% LC_{isoD}, suitably about 20% to about 30% LC_{isoD} of the total amount of LC_{D}, LC_{isoD} and LC_{succi}. In one embodiment, the present invention provides a pharmaceutical composition comprising less than about 25% of LC_{isoD} of the total amount of LC_{D}, LC_{isoD} and LC_{succi} within the first 3 months of the shelf life. In one embodiment, the present invention provides a pharmaceutical composition comprising more than about 25% of LC_{isoD} of the total amount of LC_{D}, LC_{isoD} and LC_{succi} after the 12 months of the shelf life, suitably 18 months, suitably 24 months of shelf life. In one embodiment, the present invention provides a pharmaceutical composition comprising LC_{isoD}, wherein the percentage change of LC_{isoD} over the total amount of LC_{D}, LC_{isoD} and LC_{succi} is not more than about 30%, not more than 20%, not more than 10%, suitably not more than 5% from the beginning of shelf life to the end of the shelf life, wherein shelf life is 12 months, suitably 18 months of shelf life, suitably 24 months. In one embodiment, the present invention provides a pharmaceutical composition comprising LC_{isoD}, wherein the percentage change of LC_{D} over the total amount of LC_{D}, LC_{isoD} and LC_{succi} is not more than about 20 for a period of about at least 18 months. Suitably the pharmaceutical composition is kept at about 2 to about 8° C., suitably about 5° C. The above changes can be further minimized if the pharmaceutical composition has a proper pH range. For example at the pH between about 5.5 to about 7.5, suitably at the pH between about 5.5 to about 7, suitably at the pH between about 6 to about 7, suitably about 6±0.3. Suitably the pharmaceutical formulation is kept at pH about 6.

In one embodiment, the present invention provides a pharmaceutical composition comprising at least 1% LC_{succi} of the total amount of LC_{D}, LC_{isoD} and LC_{succi} as determined by MS. In one embodiment, the present invention provides a pharmaceutical composition comprising at most 10% LC_{succi} of the total amount of LC_{D}, LC_{isoD} and LC_{succi} as determined by MS. In one embodiment, the present invention provides a pharmaceutical composition comprising from about 1% to about 10% LC_{succi} of the total amount of LC_{D}, LC_{isoD} and LC_{succi}. In one embodiment, the present invention provides a pharmaceutical composition comprising less than about 3% of LC_{succi} of the total amount of LC_{D}, LC_{isoD} and LC_{succi} within the first 3 months of the shelf life. In one embodiment, the present invention provides a pharmaceutical composition comprising more than about 5% of LC_{succi} of the total amount of LC_{D}, LC_{isoD} and LC_{succi} after the 12 months of the shelf life, suitably 18 months, suitably 24 months. In one embodiment, the present invention provides a pharmaceutical composition comprising LC_{succi}, wherein the percentage change of LC_{succi} over the total amount of LC_{D}, LC_{isoD} and LC_{succi} is not more than about 20%, not more than 10%, suitably not more than 5% from the beginning of shelf life to the end of the shelf life, wherein shelf life is 12 months, suitably 18 months, suitably 24 months. In one embodiment, the present invention provides a pharmaceutical composition comprising LC_{succi}, wherein the percentage change of LC_{succi} over the total amount of LC_{D}, LC_{isoD} and LC_{succi} is not more than about 10% for a period of about at least 18 months. Suitably the pharmaceutical composition is kept at about 2 to about 8° C., suitably about 5° C. The above changes can be further minimized if the pharmaceutical composition has a proper pH range. For example at the pH between about 5.5 to about 7.5, suitably at the pH between about 5.5 to about 7, suitably at the pH between about 6 to about 7, suitably about 6±0.3. Suitably the pharmaceutical formulation is kept at pH about 6.

In one embodiment, the present invention provides a pharmaceutical composition comprising about 60% to 80% LC_{D}, about 15% to 30% LC_{isoD} and about 1% to 10% of LC_{succi} of the total amount of LC_{D}, LC_{isoD} and LC_{succi} as determined by MS. In one embodiment, the present invention provides a pharmaceutical composition comprising about 70% to 80% LC_{D}, about 20% to 25% LC_{isoD} and about 1% to 3% of LC_{succi} of the total amount of LC_{D}, LC_{isoD} and LC_{succi} within the first 3 months of the shelf life. In one embodiment, the present invention provides a pharmaceutical composition comprising about 60% to 70% LC_{D}, about 20% to 30% LC_{isoD} and about 5% to 10% of LC_{succi} of the total amount of LC_{D}, LC_{isoD} and LC_{succi} after the 12 months of the shelf life, suitably 18 months, suitably 24 months. Suitably the pharmaceutical composition is kept at about 2 to about 8° C., suitably about 5° C. The above changes can be further minimized if the pharmaceutical composition has a proper pH range. For example at the pH between about 5.5 to about 7.5, suitably at the pH between about 5.5 to about 7, suitably at the pH between about 6 to about 7, suitably about 6±0.3. Suitably the pharmaceutical formulation is kept at pH about 6.

Thus in one embodiment, the pharmaceutical composition of the present invention further comprises a buffering system.

The present invention relates to a novel pharmaceutical composition comprising crizanlizumab, or an antibody having at most 3 amino acid difference from crizanlizumab, as active ingredient, and a buffering system, wherein the pharmaceutical composition has a pH value from about 5.0 to 7.5, from about 5.5 to 7.0, from about 5.5 to 7.5, from about 5.5 to 7.0, from about 5.5 to 6.8, from about 5.5 to 6.5, from about 5.7 to 6.8, from about 5.7 to 6.5, from about 5.7 to 6.3, from about 5.9 to 6.1, or about 6.0. In a particular aspect, the pH is any pH value within those enumerated above; for example 5.7, 5.8, 5.9, 6.0, 6.1, 6.2 and 6.3.

Suitable buffering systems for use with the invention include, but are not limited to, organic acid salts such as salts of citric acid, ascorbic acid, gluconic acid, carbonic acid, tartaric acid, succinic acid, acetic acid or phthalic acid; Tris, thomethamine hydrochloride, or phosphate buffer. Preferably, the buffering system is citric acid buffer or phosphate buffer or a combination thereof. In addition, amino acid components, e.g. glycine, can also be used as buffering agent. An amino acid may be present in its D- and/or L-form, but the L-form is typical. Preferably, the buffer system does not comprise arginine or histidine.

The concentration of the suitable buffer system used for the formulation according to the present invention is from about 10 mM to about 100 mM, from about 10 mM to about 50 mM, or from about 10 mM to about 40 mM, depending, for example, on the buffer and the desired stability of the formulation. In a preferred embodiment, the buffer system is citrate, and citrate is preferably used at a concentration from 10 to 50 mM, preferably from 15 to 40 mM, preferred from 20 to 30 mM. In another preferred embodiment, the buffer system is phosphate, and phosphate is preferably used at a concentration from 10 to 50 mM, preferably from 15 to 40 mM, preferred from 20 to 30 mM. In one embodiment, the counterion for citrate or phosphate buffer is sodium and/or potassium. In a preferred embodiment, the buffer is sodium citrate buffer.

Suitable stabilizers for use with the invention can act, e.g., as viscosity enhancing agents, solubilizing agents, isotonizing agents, and/or the like. The stabilizer can be ionic, but is preferably non-ionic (e.g. sugars). Sugars include, but are not limited to, monosaccharides, e.g., fructose, maltose, galactose, glucose, D-mannose, sorbose and the like; disaccharides, e.g. lactose, sucrose, trehalose, cellobiose, and the like; polysaccharides, e.g. raffinose, melezitose, maltodextrins, dextrans, starches, and the like; and alditols, such as mannitol, xylitol, maltitol, lactitol, xylitol sorbitol (glucitol) and the like. The sugar may be a sugar alcohol or an amino sugar. Preferably, the sugar is not a reducing sugar. Reducing sugars include, but are not limited to, all monosaccharides, lactose, maltose and cellobiose. Therefore, the sugar is preferably a non-reducing sugar, e.g. sucrose, trehalose, raffinose, sorbitol and mannitol. Sucrose is particularly useful. As ionic stabilizer they include salts such as NaCl or amino acid components. Preferably, the stabilizer does not comprise arginine or histidine. An amino acid may be present in its D- and/or L-form, but the L-form is typical. The formulation according to the present invention comprises from about 50 to 400 mM, from about 50 to 300 mM, preferably from 180 to 300 mM, most preferred about 220 mM of the stabilizer, preferably sucrose.

The pharmaceutical compositions of the invention may include, in addition to crizanlizumab or an antibody having at most 3 amino acid difference from crizanlizumab and a buffering system further components such as one or more of the following: (i) a stabilizer; (ii) a surfactant; and (iii) a salt.

Suitable surfactants according to the present invention are non-ionic surfactants, including but not limited to polysorbates (e.g. polysorbates 20 or 80); poloxamers (e.g. poloxamer 188); Triton; octyl glycoside; myristamidopropyl-, palmidopropyl-, or isostearamidopropyl-dimethylamine; polyethyl glycol, polypropyl glycol, and copolymers of ethylene and propylene glycol (e.g. Pluronics, PF68 etc). In a preferred embodiment, the surfactant is polysorbate, preferably selected from the group consisting of polysorbates 20 and polysorbates 80. More preferably, the surfactant is polysorbates 80.

The concentration of the surfactant, preferably polysorbate, used for the formulation according to the present invention is about 0.01% to 0.1%, preferably about 0.01% to 0.05%, most preferably about 0.02% weight by volume (w/v) of the formulation.

An isotonizing agent serves for setting the osmotic pressure of the formulation according to the invention to a physiologically acceptable value. The isotonizing agent is a physiologically acceptable component and is not particularly limited. Typical examples of the isotonizing agent are, for instance, an inorganic salt such as sodium chloride, potassium chloride or calcium chloride, and the like. These can be used alone or in a mixture thereof. It should be noted that some agents may have a double role, e.g., some sugars or sugar alcohols can serve both as a stabilizer and an isotonizing agent. In one embodiment, the concentration of the isotonizing agent is from about 50 mM to about 300 mM. In one embodiment, the isotonizing agent is sodium chloride. In one embodiment, sodium chloride is in the concentration from about 100 mM to about 250 mM, particularly about 190 mM.

In one embodiment, the concentration of the antibody in the pharmaceutical composition of the invention is from about 1 mg/ml to 100 mg/ml, from about 5 mg/ml to 100 mg/ml, from about 5 mg/ml to 75 mg/ml, from about 5 mg/ml to 50 mg/ml, from about 5 mg/ml to 30 mg/ml. In one embodiment, the concentration of the antibody is at least 5 mg/ml. In one embodiment, the concentration of the antibody is at least 10 mg/ml. In one embodiment, the concentration of the antibody is at least 20 mg/ml. In one embodiment, the concentration of antibody is about 10 mg/ml. Unless the context indicates otherwise, the term “antibody” or its interchanably used term “ANTIBODY”, as used herein includes crizanlizumab and any variants thereof (e.g. succinimide of crizanlizumab and iso-crizanlizumab) comprised by the pharmaceutical composition that can be detected by UV and therefore relevant in determining the protein concentration.

Furthermore, the pharmaceutical compositions of the invention are stable such that, even after storage for 36 weeks (about 9 months) at 2-8° C., less than 20%, 15%, 10%, 5%, 4%, 3%, 2%, 1% of the total antibody is aggregated as measured, for example, by SEC-HPLC. Preferably, less than 2% of the total antibody is aggregated after storage for 36 weeks (about 9 months) at 2-8° C.

The pharmaceutical compositions of the invention are stable such that, even after storage for 12 months at 2-8° C., less than 10%, 5%, 4%, 3%, 2%, 1% of the total antibody is aggregated as measured, for example, by SEC-HPLC. Preferably, less than 2% of the total antibody is aggregated after storage for 12 months at 2-8° C.

The pharmaceutical compositions of the invention are stable such that, even after storage for 15 months at 2-8° C., less than 10%, 5%, 4%, 3%, 2%, 1% of the total antibody is aggregated as measured, for example, by SEC-HPLC. Preferably, less than 2% of the total antibody is aggregated after storage for 15 months at 2-8° C.

The pharmaceutical compositions of the invention are stable such that, even after storage for 18 months at 2-8° C., less than 10%, 5%, 4%, 3%, 2%, 1% of the total antibody is aggregated as measured, for example, by SEC-HPLC. Preferably, less than 2% of the total antibody is aggregated after storage for 18 months at 2-8° C.

The pharmaceutical compositions of the invention are stable such that, even after storage for 24 months at 2-8° C., less than 10%, 5%, 4%, 3%, 2%, 1% of the total antibody is aggregated as measured, for example, by SEC-HPLC. Preferably, less than 2% of the total antibody is aggregated after storage for 24 months at 2-8° C.

The pharmaceutical compositions of the invention are stable such that, even after storage for 12 weeks (about 3 months) at 25° C., less than 5%, 4%, 3%, 2%, 1% of the total antibody is aggregated as measured by SEC-HPLC. Preferably, less than 2% of the total antibody is aggregated after storage for 12 weeks (about 3 months) at 25° C.

The pharmaceutical compositions of the invention are stable such that, even after storage for 6 months at 25° C., less than 5%, 4%, 3%, 2%, 1% of the total antibody is aggregated as measured by SEC-HPLC. Preferably, less than 2% of the total antibody is aggregated after storage for 6 months at 25° C.

The pharmaceutical compositions of the invention are stable such that, even after storage for 2 weeks at 40° C., less than 20%, 15%, 10%, 5%, 4%, 3%, 2%, 1% of the total antibody is aggregated as measured by SEC-HPLC. Preferably, less than 2% of the total antibody is aggregated after storage for 2 weeks at 40° C.

In one aspect, the invention provides pharmaceutical compositions comprising:

a) crizanlizumab and any variants thereof or an antibody having at most 3 amino acid difference from crizanlizuma and any variants thereof, used in a concentration of about 5 mg/ml to 50 mg/ml;
b) a buffering system, preferably citrate (e.g. sodium citrate) and/or phosphate (e.g. potassium phosphate) buffer systems, wherein the buffer system may be used preferably in a concentration of about 10 mM to 50 mM; and wherein the pH of the buffer system is any pH value within 5.0 to 7.5, preferably 5.5 to 7.0, preferably 5.7 to 6.3;
c) optionally, a stabilizer, preferably sucrose, preferably in a concentration of about 50 mM to 300 mM;
d) optionally, a non-ionic surfactant, preferably polysorbate 80, preferably in a concentration of about 0.01% w/v (0.1 mg/ml) to 0.1% w/v (1 mg/ml); and
e) optionally, an isotonizing agent, preferably NaCl, preferably in the concentration of about 50-300 mM, more preferably about 100-250 mM, particularly about 190 mM.

In a preferred embodiment, the present invention provides a formulation comprising SEG101 in a concentration of about 10 mg/ml, about 220 mM sucrose, about 20 mM citrate and about 0.02% w/v polysorbate 80, wherein the pH of the formulation is about 6.0, preferably from 5.7 to 6.3, more preferably from 5.9 to 6.1, e.g., 6.0.

In one embodiment, the present invention provides a pharmaceutical composition comprising crizanlizumab, iso-crizanlizumab and succinimide of crizanlizumab, e.g. as described above, wherein the pharmaceutical composition is not the formulation comprising crizanlizumab in a concentration of about 10 mg/ml, sodium phosphate about 25 mM, pH about 7, sodium chloride about 190 mM, about 0.02% w/v polysorbate 80 and water for injection.

In one aspect, the present invention provides a pharmaceutical composition comprising crizanlizumab, iso-crizanlizumab and succinimide of crizanlizumab, e.g. as described above, wherein the pharmaceutical composition does not comprise glycine.

In one aspect, the present invention provides a pharmaceutical composition comprising crizanlizumab, iso-crizanlizumab and succinimide of crizanlizumab, e.g. as described above, wherein the pharmaceutical composition does not comprise sodium phosphate.

In one aspect, the present invention provides a pharmaceutical composition comprising crizanlizumab, iso-crizanlizumab and succinimide of crizanlizumab, e.g. as described above, wherein the pharmaceutical composition does not have a pH=7.

In one aspect, the present invention provides a pharmaceutical composition comprising crizanlizumab, iso-crizanlizumab and succinimide of crizanlizumab, e.g. as described above, and at least one pharmaceutically acceptable excipients.

In one aspect, the present invention provides a pharmaceutical composition comprising crizanlizumab, iso-crizanlizumab and succinimide of crizanlizumab, e.g. as described above, wherein the pharmaceutical composition further comprises sucrose. In one embodiment the pharmaceutical composition comprises at least 50 mM sucrose.

In one aspect, the present invention provides a pharmaceutical composition comprising crizanlizumab, iso-crizanlizumab and succinimide of crizanlizumab, e.g. as described above, wherein the total amount of crizanlizumab, iso-crizanlizumab and succinimide of crizanlizumab is from about 5 mM to 50 mM, suitably from 5 mM to 30 mM, suitably 10 mM.

In one aspect, the present invention provides a pharmaceutical composition comprising crizanlizumab, iso-crizanlizumab and succinimide of crizanlizumab, e.g. as described above, wherein the pharmaceutical composition further comprises a surfactant. In one embodiment, the surfactant is polysorbate. In one embodiment, the surfactant is polysorbate 80. In one embodiment, the pharmaceutical composition comprises at least 0.01% surfactant.

In one aspect, the present invention provides a pharmaceutical composition comprising crizanlizumab, iso-crizanlizumab and succinimide of crizanlizumab, e.g. as described above, wherein the pharmaceutical composition further comprises a buffering system. In one embodiment, the buffering system is citrate buffer. In one embodiment, the pharmaceutical composition comprises 10 to 50 mM of citrate buffer.

In a preferred embodiment, the pharmaceutical composition of the present invention is in liquid form. More preferably, said compositions are aqueous, i.e. the solvent is water. A formulation or composition suitable for pharmaceutical use may be sterile, homogeneous and/or isotonic. In a preferred embodiment, the pharmaceutical compositions of the invention are suitable for intravenous administration to a human. However, the pharmaceutical compositions of the invention might not be suitable for subcutaneous administration.

In one embodiment, the pharmaceutical composition of the present invention in liquid form is also suitable for lyophilisation. Typically, the lyophilized form is more stable than the liquid form and would be the preferred form for storage. However, liquid form is preferred for its conveniencto use, if the liquid form is sufficiently stable for a period of at least 12 months, at least 18 months or at least 24 months, ideally for a period of 12 months, 18 months or 24 months.

In one embodiment, the pharmaceutical composition of the present invention is in lyophilized form, comprising crizanlizumab or an antibody having at most 3, preferably 2, more preferably only one amino acid difference from crizanlizumab, preferably crizanlizumab that has light chain and heavy chain amino acid sequences in SEQ ID NO: 10 and SEQ ID NO: 9 respectively, and a variant of crizanlizumab (iso-crizanlizumab), in which amino acid aspartic acid at position 32 of SEQ ID NO: 10 is changed to iso-aspartic acid. In one embodiment, the pharmaceutical composition further comprises succinimide of crizanlizumab.

In one embodiment, the liquid formulation subjected to lyophilization (pre-lyophilized formulation) comprises further a buffering system, wherein the pharmaceutical composition has a pH value from about 5.0 to 7.5, from about 5.5 to 7.5, from about 5.5 to 7.0, from about 5.5 to 6.8, from about 5.5 to 6.5, from about 5.7 to 6.8, from about 5.7 to 6.5, from about 5.7 to 6.3, from about 5.9 to 6.1, or about 6.0. In a particular aspect, the pH is any pH value within those enumerated above; for example 5.7, 5.8, 5.9, 6.0, 6.1, 6.2 and 6.3.

In another embodiment, the reconstituted formulation from the lyophilized formulation has a pH value from about 5.5 to 7.5, from about 5.5 to 7.0, from about 5.5 to 6.8, from about 5.5 to 6.5, from about 5.7 to 6.8, from about 5.7 to 6.5, from about 5.7 to 6.3, from about 5.9 to 6.1, or about 6.0. In a particular aspect, the pH is any pH value within those enumerated above; for example 5.7, 5.8, 5.9, 6.0, 6.1, 6.2 and 6.3.

In one embodiment, the buffering system is either a citrate buffer or a phosphate buffer. In a preferred embodiment, the buffer system is citrate, and citrate is preferably used at a concentration from 10 to 50 mM, preferably from 15 to 40 mM, preferred from 20 to 30 mM. In another preferred embodiment, the buffer system is phosphate, and phosphate is preferably used at a concentration from 10 to 50 mM, preferably from 15 to 40 mM, preferred from 20 to 30 mM.

In one embodiment, the pharmaceutical composition of the present invention in lyophilized form further comprises excipients, including but not limited to stabilizers, surfactant and isotonizing agent as taught earlier in this application. In one embodiment, the lyophilized form comprises a stabilizer. Preferably, the stabilizer is sucrose. In one embodiment, the concentration of sucrose in the pre-lyophilized formulation is between 20 to 120 mg/ml, suitably between 40 to 100 mg/ml, suitably between 60 to 90 mg/ml.

In one embodiment, the pharmaceutical composition of the present invention in lyophilized form further comprises a lyoprotectant. Typically, the lyoprotectant is added to the pre-lyophilized formulation. Lyoprotectant could be an amorphous form such as sucrose, or a crystalline form such as mannitol, or a mixture thereof. Further lyoprotectants include but not limited to glycine, polyethylene glycol and sorbitol. The concentration of the total lyoprotectants in the pre-lyophilized formulation according to the present invention is 20-150 mg/ml, suitably 40-120 mg/ml, suitably 60-120 mg/ml, suitably 60-100 mg/ml. For example, if 40 mg/ml sucrose and 60 mg/ml mannitol are in the pre-lyophilized formulation, then the concentration of the total lyoprotectants in the pre-lyophilized formulation is 100 mg/ml. In the case of an excipient having dual function, the calculation takes the total amount without distinguishing the functions. For example if the pre-lyophilized formulation comprises 40 mg/ml sucrose, then the concentration of lyoprotectant is 40 mg/ml and at the same time the concentration of stabilizer is 40 mg/ml.

In one embodiment, at least one of the lyoprotectants is sucrose. In one embodiment, the only lyoprotectant is sucrose.

In one embodiment, the lyoprotectant to antibody molar ratio is at least 300, preferably at least 500, preferably at least 600, at least 700, at least 800 or at least 900. In the case wherein at least one of the lyoprotectants is sucrose, the lyoprotectant to antibody molar ratio only refers to sucrose to antibody molar ratio. For example, in the pre-lyophilized formulation comprising 40 mg/ml of sucrose and 60 mg/ml mannitol, the molar ratio of lyoprotectant over antibody is 553, which does not take into account the amount of mannitol as lyoprotectant. In the calculation of antibody all charge variants, including the main peak, are taken together, which is preferably determined by UV. For example, in the case the pre-lyophilized formulation contains 30 mg/ml SEG101, this refers to the total charge variants, including the main peak (crizanlizumab).

In one embodiment, the concentration of the antibody in pre-lyophilized formulation is from about 10 mg/ml to 100 mg/ml, from about 10 mg/ml to 70 mg/ml, from about 20 mg/ml to 70 mg/ml, from about 30 mg/ml to 50 mg/ml, e.g. about 40 mg/ml.

Using a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution.

The surfactant can be added to the pre-lyophilized formulation, the lyophilized formulation and/or the reconstituted formulation as desired, suitably to the pre-lyophilized formulation.

In one embodiment, the surfactant is a non-ionic surfactant. In one embodiment, the surfactant is a polysorbate, preferably poylsorbate 80 or poylsorbate 20. Preferably, the surfactant is in a concentration of 0.01% w/v (0.1 mg/mL) to 0.1% w/v (1 mg/mL), preferably 0.01% w/v (0.1 mg/mL) to 0.05% w/v (0.5 mg/mL), preferably 0.02% w/v (0.2 mg/mL).

Ideally, the lyophilized form is stored at room temperature. Alternatively, the lyophilized form is stored at 2-8° C. Ideally, the lyophilized form has a shelf life of at least 18 months, at least 24 months or at least 36 months. Ideally, the lyophilized form has a shelf life of 18 months, 24 months or 36 months.

The lyophilized formulation can be reconstituted shortly prior to administration to patients. The reconstitution is achieved within acceptable period of time, typically less than 10 minutes. Reconstitution results in lower, same or higher antibody concentration, compared to the pre-lyophilized formulation, but commonly in lower antibody concentration. The reconstituted formulation retains essentially the physical and chemical stability and integrity upon storage for a period of time from the reconstitution to the use, typically a few hours and up to several days. The reconstitution medium is selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 50 mM to about 100 mM. The most preferred reconstitution medium is sterile water. The reconstituted formulation can achieve the required isotonicity by dilution of the reconstituted formulation with an infusion solution before administration.

In one aspect the present invention provides a lyophilized formulation obtainable by lyophilizing an aqueous formulation having a pH value from about 5.0 to 7.5, preferably 5.5 to 7.5, wherein the lyophilized formulation comprises:

a) antibody (crizanlizumab and any variants thereof);
b) a lyoprotectant; and
c) a buffer system.

In one embodiment the lyophilized formulation further comprises a surfactant, preferably polysorbate 40 or polysorbate 80. Preferably surfactant is present in the aquous formulation in a concentration of about 0.01% w/v (0.1 mg/mL) to 0.1% w/v (1 mg/mL).

In one embodiment the antibody is present in the aqueous formulation in a concentration of about 10 mg/mL to 100 mg/mL.

In one embodiment the buffer system is citrate, e.g. sodium citrate. Preferably the buffer system is present in the aqueous formulation in a concentration of about 10 mM to 50 mM. In one embodiment the lyoprotectant is sucrose, mannitol or a mixture thereof. Preferably the lyoprotectant is present in the aqueous formulation in a concentration of about 10 mg/mL to 100 mg/mL.

In one embodiment the molar ratio of lyoprotectant, preferably sucrose, to antibody is from about 200 to 1500.

In one embodiment the present invention provides a lyophilized formulation comprising

a) about 25-40 w/w %, preferably about 28% to 32 w/w %, of antibody; and
b) about 55% -75 w/w %, preferably about 65% to 71 w/w % of sucrose, based on the total weight of the lyophilized formulation.

In one embodiment, the lyophilized formulation is obtainable from lyophilizing an aqueous formulation, wherein the aqueous formulation has a pH of about 5.7 to 6.3 and comprises

a) crizanlizumab and any variants thereof in a concentration of about 30 mg/mL to about 50 mg/mL;
b) sucrose in a concentration of about 10 mg/mL to 100 mg/mL;
c) optionally, mannitol in a concentration of up to 100 mg/mL;
d) citrate (e.g. sodium citrate) in a concentration of about 20 mM; and
e) polysorbate 80 in a concentration of about 0.02% w/v (0.2 mg/mL).

In one embodiment the lyophilized formulation is obtainable from lyophilizing an aqueous formulation, wherein the aqueous formulation has a pH of about 5.7 to 6.3, preferably about pH 6.0, and comprises

a) crizanlizumab and any variants thereof in a concentration of about 30 mg/mL;
b) sucrose in a concentration of about 90 mg/mL;
c) citrate (e.g. sodium citrate) in a concentration of about 20 mM; and
d) polysorbate 80 in a concentration of about 0.02% w/v (0.2 mg/mL).

In one embodiment the lyophilized formulation is obtainable from lyophilizing an aqueous formulation, wherein the aqueous formulation has a pH of about 5.7 to 6.3, preferably about pH 6.0, and comprises

a) crizanlizumab and any variants thereof in a concentration of about 40 mg/mL;
b) sucrose in a concentration of about 90 mg/mL;
c) citrate (e.g. sodium citrate) in a concentration of about 20 mM; and
d) polysorbate 80 in a concentration of about 0.02% w/v (0.2 mg/mL).

In one embodiment the lyophilized formulation is obtainable from lyophilizing an aqueous formulation, wherein the aqueous formulation has a pH of about 5.7 to 6.3, preferably about pH 6.0, and comprises

a) crizanlizumab and any variants thereof in a concentration of about 50 mg/mL;
b) sucrose in a concentration of about 40 mg/mL;
c) citrate (e.g. sodium citrate) in a concentration of about 20 mM; and
d) polysorbate 80 in a concentration of about 0.02% w/v (0.2 mg/mL).

In one embodiment the lyophilized formulation is obtainable from lyophilizing an aqueous formulation, wherein the aqueous formulation has a pH of about 5.7 to 6.3, preferably about pH 6.0, and comprises

a) crizanlizumab and any variants thereof in a concentration of about 30 mg/mL;
b) sucrose in a concentration of about 20 mg/mL;
c) mannitol in a concentration of about 40 mg/mL;
d) citrate (e.g. sodium citrate) in a concentration of about 20 mM; and
e) polysorbate 80 in a concentration of about 0.02% w/v (0.2 mg/mL).

In one embodiment the lyophilized formulation is obtainable from lyophilizing an aqueous formulation, wherein the aqueous formulation has a pH of about 5.7 to 6.3, preferably about pH 6.0, and comprises

a) crizanlizumab and any variants thereof in a concentration of about 30 mg/mL;
b) sucrose in a concentration of about 40 mg/mL;
c) mannitol in a concentration of about 80 mg/mL;
d) citrate (e.g. sodium citrate) in a concentration of about 20 mM; and
e) polysorbate 80 in a concentration of about 0.02% w/v (0.2 mg/mL).

In one embodiment the lyophilized formulation is obtainable from lyophilizing an aqueous formulation, wherein the aqueous formulation has a pH of about 5.7 to 6.3, preferably about pH 6.0, and comprises

a) crizanlizumab and any variants thereof in a concentration of about 30 mg/mL;
b) sucrose in a concentration of about 40 mg/mL;
c) mannitol in a concentration of about 60 mg/mL;
d) citrate (e.g. sodium citrate) in a concentration of about 20 mM; and
e) polysorbate 80 in a concentration of about 0.02% w/v (0.2 mg/mL).

In one aspect the present invention provides a liquid pharmaceutical composition obtained by reconstituting the lyophilized formulation as described above.

It is an object of the present invention to provide an antibody formulation which is stable during storage time. According to the present invention, a stable formulation is a formulation wherein the antibody therein essentially retains its potency and physical and chemical stability and integrity upon storage. The stability of the antibody formulation may be measured using biological activity assays. Preferably, the reduction in the activity of the antibody is less than 20%, more preferably less than 15%, more preferably less than 10%, more preferably less than 5% upon storage in long-term storage conditions (2-8° C.) for 24 or 36 weeks, or for 12 months, 15 months, 18 months or 24 months, as compared to the starting time (i.e. T=0). As far as SEG101 is concerned, the biological activity is determined based on its ability to inhibit the interaction of cells expressing human P-selectin with its ligand PSGL-1, for example mammalian cells recombinantly modified to present human P-selectin on their surface, with recombinant human PSGL-1. Suitably it is determined by measuring the fluorescence signal from the microtiter plates whose wells are first coated with PSGL-1 and then incubated with mammalian cells expressing human P-selection on their surface, after the cells are fluorescently labelled and incubated with SEG101 comprised in the pharmaceutical composition of the present invention. A suitable way of measuring the biological activity of SEG101 or its variants is demonstrated in Example 2.1.

In one embodiment, the pharmaceutical composition of the invention exhibits undetectable or only very low levels of antibody aggregation during long periods of storage as described above. In preferred embodiments, at least 80%, at least 85%, at least 90%, at least 95%, at least 96%, at least 97%, at least 98% or at least 99% of the antibody molecules in the formulation are present as monomers, as e.g. determined by size-exclusion chromatography (SEC), after storage in 2-8° C. for 6 weeks, for 12 weeks, for 24 weeks, for 36 weeks, for 48 weeks, for one year or for 18 months or for 24 months. Most preferably, at least 97% of antibody molecules in the formulation are present as monomers after storage in 2-8° C. for 36 weeks or for one year, for 18 months or for 24 months. Preferably, SEC is performed as described in example 3.

Preferably, a liquid antibody formulation should exhibit a shelf life of 6 months or more. The pharmaceutical composition of the present invention exhibits a shelf life of at least 9 months, e.g. 9 months, at least one year, e.g. 1 year, at least 18 months, e.g. 18 months, or up to 2 year, e.g. 24 months, preferably when kept at 2-8° C. Preferably, the pharmaceutical composition exhibits a shelf life of about 12 months, 18 months or suitably 24 months. The main factors determining shelf life usually are formation of by-products and degradation products and loss of bioactivity. During its shelf life, the biological activity of crizanlizumab should remain between 80% and 125% of the original activity. The formulation of the current invention achieves these desired stability levels.

### Anti-P-Selectin Antibodies

Antibodies against human P-selectin are known from, e.g. WO 2008/069999, and include antibodies which are characterized by comprising heavy chain CDR1, CDR2 and CDR3 of SEQ ID NOs 1, 2 and 3, and light chain CDR1, CDR2 and CDR3 of SEQ ID NOs 4, 5 and 6, respectively. Antibodies against human P-selectin may also be characterized by comprising a VH domain with the amino acid sequence of SEQ ID NO: 7 and a VL domain with the amino acid sequence of SEQ ID NO: 8. In particular, SEG, is a humanised monoclonal antibody against P-selectin comprising a heavy chain of SEQ ID NO: 9 and light chain of SEQ ID NO: 10. The antibody binds to the lectin-binding domain located in the amino terminus of P-selectin with high affinity and specificity and blocks the interaction of P-selectin with its receptor P-selectin glycoprotein ligand-1 (PSGL-1).

Crizanlizumab is a humanised monoclonal antibody directed against human P-selectin, and is also described in reference WO 2018/083645 A1. Crizanlizumab is characterized by comprising a heavy chain of SEQ ID NO: 9 and light chain of SEQ ID NO: 10. Table 1 summarizes the sequence characteristics of SEG101.

TABLE 1
Amino acid sequence specifications of SEG101.

| SEQ ID | | |
|---|---|---|
| NO. | Description | Sequence |
| 1 | Heavy chain CDR1 | SYDIN |
| 2 | Heavy chain CDR2 | WIYPGDGSIKYNEKFKG |
| 3 | Heavy chain CDR3 | RGEYGNYEGAMDY |
| 4 | Light chain CDR1 | KASQSVDYDGHSYMN |
| 5 | Light chain CDR2 | AASNLES |
| 6 | Light chain CDR3 | QQSDENPLT |
| 7 | Heavy chain | QVQLVQSGAEVKKPGASVKVSCKVSGYTFTSYDINWVRQA |
| | variable region | PGKGLEWMGWIYPGDGSIKYNEKFKGRVTMTVDKSTDTAY |
| | (V_{H}) | MELSSLRSEDTAVYYCARRGEYGNYEGAMDYWGQGTLVTV |
| | | SS |
| 8 | Light chain | DIQMTQSPSSLSASVGDRVTITCKASQSVDYDGHSYMNWY |
| | variable | QQKPGKAPKLLIYAASNLESGVPSRFSGSGSGTDFTLTIS |
| | region (V_{L}) | SLQPEDFATYYCQQSDENPLTFGGGTKVEIKR |
| 9 | Heavy chain | QVQLVQSGAEVKKPGASVKVSCKVSGYTFTSYDINWVRQA |
| | | PGKGLEWMGWIYPGDGSIKYNEKFKGRVTMTVDKSTDTAY |
| | | MELSSLRSEDTAVYYCARRGEYGNYEGAMDYWGQGTLVTV |
| | | SSASTKGPSVFPLAPCSRSTSESTAALGCLVKDYFPEPVT |
| | | VSWNSGALTSGVHTFPAVLQSSGLYSLSSVVTVTSSNFGT |
| | | QTYTCNVDHKPSNTKVDKTVERKCCVECPPCPAPPVAGPS |
| | | VFLFPPKPKDTLMISRTPEVTCVVVDVSHEDPEVQFNWYV |
| | | DGMEVHNAKTKPREEQFNSTFRVVSVLTVVHQDWLNGKEY |
| | | KCAVSNKGLPAPIEKTISKTKGQPREPQVYTLPPSREEMT |
| | | KNQVSLTCLVKGFYPSDIAVEWESNGQPENNYKTTPPMLD |
| | | SDGSFFLYSKLTVDKSRWQQGNVFSCSVMHEALHNHYTQK |
| | | SLSLSPGK |
| 10 | Light chain | DIQMTQSPSSLSASVGDRVTITCKASQSVDYDGHSYMNWY |
| | | QQKPGKAPKLLIYAASNLESGVPSRFSGSGSGTDFTLTIS |
| | | SLQPEDFATYYCQQSDENPLTFGGGTKVEIKRTVAAPSVF |
| | | IFPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |

### Target Diseases and Disorders

The pharmaceutical compositions of the invention can be used to treat, ameliorate or prevent a variety of diseases or disorders. Pharmaceutical compositions of the invention are particularly useful to treat P-selectin mediated or P-selectin related disorders, in particular to reduce or eliminate vaso-occlusion, inflammation, and pain crises associated with sickle cell disease; see for instance WO 2018/083645A1 and WO 2008/069999A2.

In the context of the present invention, the term “P-selectin mediated disorder” or “P-selectin related disorder” refers to a disorder which is associated with or characterized by increased levels of P-selectin/PSGL-1 complexes. An anti-P-selectin antibody or binding fragment thereof can have the ability to reduce the formation of P-selectin/PSGL-1 complexes. They may also have the ability to dissociate pre-formed P-selectin/PSGL-1 complexes. Accordingly, it will be appreciated that the use of pharmaceutical compositions of the invention comprising anti-P-selectin antibodies or binding fragments thereof allows the prevention of P-selectin mediated disorders by inhibiting the formation of new P-selectin/PSGL-1 complexes. It will also be appreciated that the use of said formulations allows the treatment of existing P-selectin mediated disorders by dissociating pre-formed P-selectin/PSGL-1 complexes. Suitably, the reduction in the formation of P-selectin/PSGL-1 complexes and the dissociation of such complexes occurs during cell to cell interactions. Therefore, suitably disorders prevented by the anti-P-selectin antibodies or binding fragments thereof described herein are disorders associated with increased levels of P-selectin/PSGL-1 complexes in cell to cell interactions.

Increased levels of P-selectin/PSGL-1 complexes may be observed in a wide range of disorders and/or symptoms. In particular, they are observed in subjects or samples from subjects with inflammatory and/or thrombotic disorders and/or symptoms. Thus, pharmaceutical compositions of the invention comprising an anti-P-selectin antibody or binding fragment thereof may be used to treat disorders, such as inflammatory and/or thrombotic disorders selected from the group consisting of: sickle cell disease, sickle cell pain crises, arthritis (e.g., rheumatoid arthritis, osteoarthritis, and psoriatic arthritis), graft rejection, graft versus host disease, asthma, chronic obstructive pulmonary disease, psoriasis, dermatitis, sepsis, nephritis, lupus erythematosus, scleroderma, rhinitis, anaphylaxis, diabetes, multiple sclerosis, atherosclerosis, thrombosis, tumour metastasis, allergic reactions, thyroiditis, ischemic reperfusion injury (e.g., due to myocardial infarction, stroke, or organ transplantation), cancer (e.g., multiple myeloma) and conditions associated with extensive trauma, or chronic inflammation, such as, for example, type IV delayed hypersensitivity, associated for example with infection by Tubercle bacilli, or systematic inflammatory response syndrome, or multiple organ failure.

Sickle cell pain crises may be experienced by subjects with sickle cell disease. The pharmaceutical compositions of the invention comprising anti-P-selectin antibody or binding fragment thereof may have particular utility in treating and preventing vaso-occlusive pain crisis in sickle cell disease in a subject. Suitably, subjects with sickle cell disease has a genotype selected from the group consisting of: HbSS, HbSC, HbSO-thalassemia and HbSβ0+ thalassemia.

### Patient Administration

It is an object of the present invention to provide a use of the formulation of the invention for the treatment of P-selectin mediated diseases or medical conditions. It provides a method of treating P-selectin mediated diseases or medical conditions, suitably sickle cell disease, comprising the step of administering the pharmaceutical composition of the invention into a subject in need thereof. Pharmaceutical composition of the invention are useful for the prophylaxis and treatment of P-selectin mediated diseases or medical conditions, e.g. inflammatory and thrombotic diseases, tumour metastasis, and in particular to reduce or eliminate vaso-occlusion, inflammation, and pain crises associated with sickle cell disease, and preferably with the dosing regimens described in WO 2018/083645A1, from page 13, third paragraph to page 19, third paragraph, said pages being hereby incorporated by reference. Formulations of the invention may be administered as the sole treatment or in conjunction with other drugs or therapies useful in treating the conditions as described herein before.

In one aspect the invention relates to the pharmaceutical composition for use in the treatment and/or prevention of P-selectin mediated diseases or medical conditions, for example in the prevention of a sickle cell pain crisis, wherein the composition is first provided in a loading phase, during which the subject receives two loading doses of the antibody at an amount of 5 mg/kg to 7.5 mg/kg and wherein the time interval between the two loading doses is 2 weeks (+/−3 days), and then further provided in a maintenance phase, during which the subject receives a plurality of maintenance doses of the antibody at an amount of 5 mg/kg to 7.5 mg/kg and wherein the time interval between the plurality of maintenance doses is 4 weeks.

In one aspect, the pharmaceutical composition of the present invention is in a vial.

In one aspect, the pharmaceutical composition of the present invention is administered to a patient by intravenous route, typically by transferring it via a syringe into an infusion container (e.g. an infusion bag made from plastics) filled with an isotonic solution (e.g. 0.9% NaCl or 5% dextrose) with which infusion is conducted.

In one embodiment, the pharmaceutical composition of the present invention is administered to a subject, typically a healthy volunteer or a patient, preferably by intravenous route, preferably in an amount of 5 mg/kg, preferably with 2 loading doses 2 weeks apart, followed by a plurality of maintenance doses every 4 weeks. In one embodiment, the concentration of crizanlizumab and any variants thereof in serum is determined, and one or more or all of the following PK parameters are met:

a) a tₘₐₓ in the range from 0.4 to 10 hours (h), preferably from 0.55 h to 6.25 h, with a preferred Median of 1.5 to 2.5 h, preferably of 1.92 h, after administration of said pharmaceutical composition;
b) a Cₘₐₓ in the range of, after first dose, 116±91.3 μg/mL; or preferably at steady state at 50 to 200 μg/mL, preferably at 124±31.6 μg/mL;
c) an apparent t_{1/2} in the range of 100 h to 300 h, preferably in the range of 150 h to 210 h, e.g. at about 183 h (7.6 days);
d) an AUC_{tau, ss} in the range of 10 000 to 30 000 μg×h/mL, preferably at week 15 at 20400 μg×h/mL, preferably with a Coefficient of Variance of 23.5%. “tau” here refers to the dosing interval, so AUCtau, steady state (ss) is the AUC from the beginning of infusion at week 15 day 1 to right before the infusion at week 19 day;
e) a mean clearance at steady state week 15, in a patient with SCD and typically a body weight of 70 kg, in the range of 10 to 30 mL/h, preferably 15 to 20 mL/h, e.g. at about 17.2 mL/h;
f) a PK trough concentration obtained every 4 weeks in the steady state, especially from week 7 to week 27, in the range from about 3.78 μg/mL to 9.8 μg/mL. In addition at week 3, two weeks after the previous infusion the trough concentration is about 12 μg/mL to about 24 μg/mL, preferably about 15 μg/mL to about 21 μg/mL, preferably about 18.2 μg/mL.

The concentration of crizanlizumab and its variants in serum is determined typically ex vivo and typically by ELISA, typically using P-selectin as bait. Hence the method measures all serum crizanlizumab and its variants that are capable of binding to P-selectin and preferably that can be further bound by an anti-human IgG antibody used in the assay.

Typically a sandwich ELISA is used to detect crizanlizumab and any variants thereof in human serum. A high-bind immunoplate was coated with a mouse anti-human P-selectin antibody. The plate was then coated with human P-selectin. Quantified crizanlizumab and any variants thereof was used to prepare standards and quality control (QC) samples and then added to designated sample wells. The amount of bound crizanlizumab is visualized by the subsequent additions of biotinylated goat anti-human IgG, streptavidin protein that is covalently conjugated to horseradish peroxidase (streptavidin-HRP), and a chromogenic substrate, tetramethylbenzidine (TMB), and the product of this reaction were detected at 450 nm with a spectrophotometer. The concentration of crizanlizumab and variants thereof in serum samples is back-calculated from the standard calibration curve.

In one embodiment, the pharmaceutical composition of the present invention is administered to a subject, typically a healthy volunteer or a patient, preferably by intravenous route, preferably in an amount of 5 mg/kg, preferably with 2 loading doses 2 weeks apart, followed by a plurality of maintenance doses every 4 weeks, wherein crizanlizumab and variants thereof in serum achieves at least 70%, at least 80%, at least 90%, at least 95% inhibition of binding of P-selectin to PSGL-1, typically determined by Surface Plasmon Resonance (SPR) assay.

Surface Plasmon Resonance (SPR) assay is an in vitro binding competition assay. In the absense of crizanlizumab and any variants thereof, the binding of P-selectin to its target PSGL-1 yields quantifiable signal, set as 100%. The addition of crizanlizumab and any variants thereof, for example by adding the pharmaceutical composition of the invention, or by adding the serum obtained from a human subject who has received the pharmaceutical composition of the invention, inhibits binding of P-Selectin to PSGL-1, resulting in reduced signal, which can be calculated into percentage of inhibition.

In one embodiment of the SPR assay, P-selectin is substituted by P-selectin fused to immunoglobulin (PSel-Ig) and PSGL-1 by glycosulfopeptide 6 (GSP6), which represents a minimal peptide version of PSGL-1, still capable of binding PSel-Ig.

A further SPR embodiment is delineated in EXAMPLE 6.

In one aspect the pharmaceutical composition of the present invention comprises crizanlizumab and iso-crizanlizumab, wherein the IC50 determined by using the pharmaceutical composition is in the range of about 4-7 μg/ml, typically about 4.6-6.2 μg/ml, typically about 5.0 to 5.7 μg/ml, typically about 5.2 μg/ml. In one embodiment the IC50 is determined in an in vitro assay.

### EXAMPLES

### Example 1: Preparation of Liquid Formulations for SEG101

SEG101 can be produced, for example, by the methods described in WO 2008/069999, from page 15, line 20 to page 18, line 29, which pages are herein incorporated by reference. Table 2 shows formulations tested for their suitability for SEG101. Samples B and C, and samples F and G, respectively are identical to evaluate variability within the formulation.

TABLE 2
Tested formulations.

| Sample | SEG101 | | | Stabilizing/isotonizing | Polysorbate |
|---|---|---|---|---|---|
| name | (mg/mL) | Buffer | pH | agent | 80 |
| A | 10 | Sodium Phosphate | 7.0 | 190 mM NaCl | 0.02% |
| | | (25 mM) | | | |
| B | 10 | Sodium Phosphate | 7.0 | 220 mM sucrose | 0.02% |
| | | (25 mM) | | | |
| C | 10 | Sodium Phosphate | 7.0 | 220 mM sucrose | 0.02% |
| | | (25 mM) | | | |
| D | 50 | Sodium Phosphate | 7.0 | 220 mM sucrose | 0.02% |
| | | (25 mM) | | | |
| E | 10 | Sodium Phosphate | 7.0 | 150 mM L-Arginine HCl | 0.02% |
| | | (25 mM) | | | |
| F | 10 | Histidine (20 mM) | 6.0 | 220 mM sucrose | 0.02% |
| G | 10 | Histidine (20 mM) | 6.0 | 220 mM sucrose | 0.02% |
| H | 50 | Histidine (20 mM) | 6.0 | 220 mM sucrose | 0.02% |
| I | 10 | Histidine (20 mM) | 6.0 | 150 mM L-Arginine HCl | 0.02% |
| J | 10 | Sodium Phosphate | 6.0 | 220 mM sucrose | 0.02% |
| | | (20 mM) | | | |
| K | 10 | Sodium Citrate (20 mM) | 6.0 | 220 mM sucrose | 0.02% |

### 1 Results

### 1.1 Potency Assay

For all samples, potency measured by ELISA and cell-based assay was at start of the stability study within expectations. Current specifications for ELISA were 80%-125% relative biological activity compared to the reference substance.

Potency assay by binding ELISA did not show relevant changes for any of the samples or stability time points measured (Table 3). In cell-based assay (Table 4) a significant reduction of potency was observed at 40° C./2 weeks as well as 25° C./12 weeks for all Histidine and/or Arginine containing samples (Table 3). Samples in phosphate or citrate buffer did not show this reduction in potency.

In a separate experiment for formulation K, potency was measured by ELISA and cell-based assay (using the same methods as for the other formulations) in different time points (Table 5).

TABLE 3
Potency by Binding ELISA (binding to P-selectin)

| | | T = 2 | T = 12 | T = 12 | T = 24 | T = 36 |
|---|---|---|---|---|---|---|
| | | weeks at | weeks at | weeks at | weeks at | weeks at |
| | T = 0 | 40° C. | 2-8° C. | 25° C. | 2-8° C. | 2-8° C. |
| | [% rel. | [% rel. | [% rel. | [% rel. | [% rel. | [% rel. |
| | biological | biological | biological | biological | biological | biological |
| Sample | activity] | activity] | activity] | activity] | activity] | activity] |
| A: pH 7, NaCl, | 105 | 87 | 105 | 93 | NT | NT |
| phosphate 10 mg/mL | | | | | | |
| D: pH 7, Sucrose, | 106 | 97 | 94 | 84 | NT | NT |
| phosphate 50 mg/mL | | | | | | |
| E: pH 7, Arg. HCl, | 111 | 85 | NT | NT | NT | NT |
| phosphate, 10 mg/mL | | | | | | |
| F: pH 6, Sucrose, | 103 | 97 | 102 | 89/83* | 103 | 106 |
| Histidine, 10 mg/mL | | | | | | |
| H: pH 6, Sucrose, | 103 | 90 | 102 | 92 | NT | NT |
| Histidine, 50 mg/mL | | | | | | |
| I: pH 6, Arg. HCl, | 107 | 92 | NT | NT | NT | NT |
| Histidine, 10 mg/mL | | | | | | |
| J: pH 6, Sucrose, | NT | 110 | 104 | 101/104* | 110 | 103 |
| Na-phosphate, | | | | | | |
| K: pH 6, Sucrose, | NT | 109 | 118 | 102/101* | 112 | 108 |
| Citrate, 10 mg/mL | | | | | | |

NT: Not Tested
*Re-analysis

TABLE 4
Potency by cell-based assay

| | | T = 2 | T = 12 | T = 12 | T = 24 | T = 36 |
|---|---|---|---|---|---|---|
| | | weeks at | weeks at | weeks at | weeks at | weeks at |
| | T = 0 | 40° C. | 2-8° C. | 25° C. | 2-8° C. | 2-8° C. |
| | [% rel. | [% rel. | [% rel. | [% rel. | [% rel. | [% rel. |
| | biological | biological | biological | biological | biological | biological |
| Sample name | activity] | activity] | activity] | activity] | activity] | activity] |
| A: pH 7, NaCl, | 114 | 89 | 107 | 96 | NT | NT |
| phosphate 10 mg/mL | | | | | | |
| D: pH 7, Sucrose, | 102 | 82 | 105 | 89 | NT | NT |
| phosphate 50 mg/mL | | | | | | |
| E: pH 7, Arg. HCl, | 98 | 71 | NT | NT | NT | NT |
| phosphate, 10 mg/mL | | | | | | |
| F: pH 6, Sucrose, | 105 | 70 | 90 | 63/57* | 93 | 101 |
| Histidine, 10 mg/mL | | | | | | |
| H: pH 6, Sucrose, | 91 | 72 | 98 | 73 | NT | NT |
| Histidine, 50 mg/mL | | | | | | |
| I: pH 6, Arg. HCl, | 101 | 67 | NT | NT | NT | NT |
| Histidine, 10 mg/mL | | | | | | |
| J: pH 6, Sucrose, | NT | 91 | 105 | 91/79* | 101 | 108 |
| Na- phosphate, | | | | | | |
| K: pH 6, Sucrose, | NT | 87 | 104 | 96/85* | 97 | 101 |
| Citrate, 10 mg/mL | | | | | | |

NT: Not Tested
*Re-analysis

TABLE 5
Potency by ELISA and cell-based assay for the formulation K.

| | Binding to P- | Inhibition of adhesion of P-selectin |
|---|---|---|
| | selectin by | expressing mammalian cells to |
| | ELISA [%] | PSGL-1 [%] |
| Initial analysis (T = 0) | 100 | 99 |
| 5° C./ambient RH | | |
| 1.5 months | 99 | 100 |
| 3 months | 95 | 99 |
| 6 months | 81 | 93 |
| 9 months | 100 | 105 |
| 12 months | 99 | 95 |
| 15 months | 96 | 92 |

RH: relative humidity.

The samples were kept in sealed vials. The humidity outside of the vials is in practice not relevant for the stability of the drug.

### 1.2 Charge Variants by CZE

There were only very limited changes in the charge variant profile over time at the long-term storage condition (2-8° C.). However, changes are very pronounced at accelerated (25° C.) and stressed (40° C.) conditions.

### 1.2.1 Impact of pH Value on Degradation Pathway

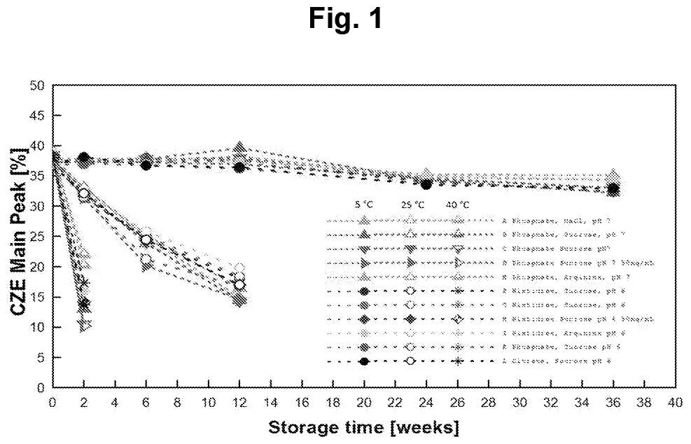

Changes in CZE Main peak appear mainly temperature-driven, and no relevant differences are observed between pH 6.0 and pH 7.0 set point (FIG. 1, Table 6).

TABLE 6
Impact of formulation pH on CZE main peak. Values in
columns A-K represent the main peak % AUC in CZE diagram.

| | A | B | C | D | E | F | G | H | I | J | K |
|---|---|---|---|---|---|---|---|---|---|---|---|
| Initial | 38.2 | 38.3 | 38.4 | 38.1 | 39.0 | 37.9 | 37.7 | 38.3 | 38.1 | 38.1 | 38.0 |
| analysis | | | | | | | | | | | |
| (T = 0) | | | | | | | | | | | |
| 5° C./ | | | | | | | | | | | |
| ambient | | | | | | | | | | | |
| RH | | | | | | | | | | | |
| 2 weeks | 37.3 | 37.8 | 37.6 | 37.4 | 37.7 | 37.0 | 36.9 | 37.4 | 37.8 | 37.1 | 38.1 |
| 6 weeks | 37.3 | 37.8 | 37.8 | 37.2 | 37.9 | 37.9 | 37.7 | 37.3 | 36.9 | 36.7 | 36.7 |
| 12 weeks | 38.2 | 39.6 | 37.9 | 36.9 | 37.6 | 37.3 | 37.7 | 37.4 | 37.3 | 36.2 | 36.4 |
| 6 months | 34.8 | 34.3 | 34.6 | 34.5 | 35.2 | 34.0 | 34.2 | 34.1 | 35.0 | 33.7 | 33.5 |
| 9 months | 34.3 | 32.2 | 33.0 | 33.0 | 35.1 | 32.4 | 32.3 | 32.4 | 34.4 | 33.0 | 32.9 |
| 25° C./60% | | | | | | | | | | | |
| RH | | | | | | | | | | | |
| 2 weeks | 33.0 | 32.5 | 32.1 | 31.5 | 33.1 | 31.5 | 31.3 | 31.6 | 32.2 | 31.5 | 32.1 |
| 6 weeks | 24.8 | 23.8 | 24.0 | 24.5¹ | 25.2 | 24.6 | 25.1 | 24.4 | 25.8 | 24.9¹ | 24.4 |
| 12 weeks | 14.9 | 14.5 | 14.3 | 14.6 | 16.4 | 18.1 | 18.4 | 17.0 | 19.8 | 16.8 | 17.0 |
| 40° C./75% | | | | | | | | | | | |
| RH | | | | | | | | | | | |
| 2 weeks | 22.0 | 13.1 | 10.5 | 10.2 | 20.3 | 17.3 | 13.7 | 14.2 | 16.0 | 13.7 | 13.8 |

RH: relative humidity.
¹Main peak including basic 0 peak.

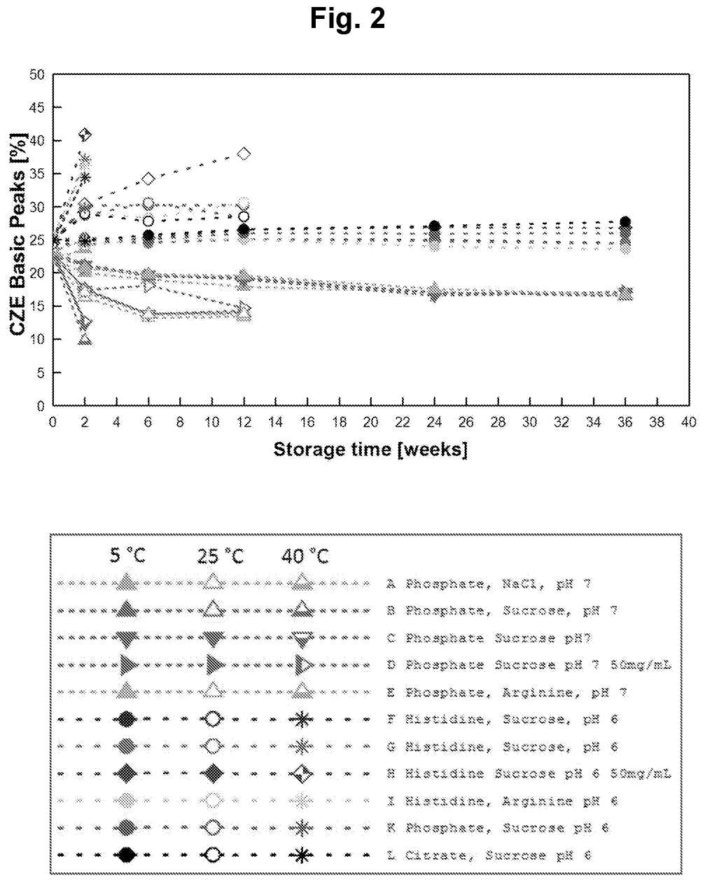

Compared to the starting material, basic variants increased at pH 6.0, while at pH 7.0 a decrease over time is observed (FIG. 2, Table 7). At 2-8° C. at pH 6.0, the level of basic peaks stayed almost constant, while at pH 7.0 at 2-8° C. a slow but constant decrease was observed. At accelerated conditions (25° C.), both at pH 6.0 and 7.0, a plateau appears to be reached.

TABLE 7
Impact of formulation pH on CZE basic peaks. Values in columns
A-K represent the sum of basic variants’ % AUC in CZE diagram.

| | A | B | C | D | E | F | G | H | I | J | K |
|---|---|---|---|---|---|---|---|---|---|---|---|
| Initial | 22.1 | 22.6 | 23.2 | 23.0 | 23.3 | 24.8 | 24.6 | 24.5 | 25.4 | 25.1 | 24.9 |
| analysis | | | | | | | | | | | |
| (T = 0) | | | | | | | | | | | |
| 5°C/ | | | | | | | | | | | |
| ambient | | | | | | | | | | | |
| RH | | | | | | | | | | | |
| 2 weeks | 20.0 | 21.1 | 21.2 | 20.9 | 20.7 | 25.0 | 24.9 | 24.2 | 24.1 | 25.3 | 24.9 |
| 6 weeks | 19.0 | 19.5 | 19.8 | 19.6 | 19.8 | 24.6 | 24.5 | 25.3 | 25.3 | 25.1 | 25.7 |
| 12 weeks | 17.9 | 19.2 | 19.0 | 19.3 | 19.6 | 25.1 | 25.1 | 26.5 | 25.0 | 26.0 | 26.6 |
| 6 months | 17.4 | 17.3 | 18.0 | 17.8 | 17.9 | 25.4 | 25.2 | 27.2 | 24.3 | 26.3 | 27.6 |
| 9 months | 16.6 | 16.5 | 17.1 | 17.1 | 16.7 | 24.5 | 24.4 | 26.8 | 23.6 | 26.0 | 27.7 |
| 25° C./60% | | | | | | | | | | | |
| RH | | | | | | | | | | | |
| 2 weeks | 16.3 | 17.6 | 17.4 | 17.4 | 17.1 | 30.2 | 30.1 | 30.4 | 29.1 | 28.7 | 28.9 |
| 6 weeks | 13.2 | 13.8 | 13.8 | 13.8 | 13.7 | 30.2 | 30.3 | 34.2 | 28.3 | 26.8 | 27.8 |
| 12 weeks | 13.4 | 14.0 | 14.3 | 14.7 | 13.8 | 30.2 | 30.3 | 38.0 | 30.6 | 28.4 | 28.5 |
| 40° C./75% | | | | | | | | | | | |
| RH | | | | | | | | | | | |
| 2 weeks | 23.6 | 9.8 | 12.3 | 12.7 | 25.0 | 34.4 | 37.1 | 40.9 | 36.2 | 29.8 | 28.4 |

RH: relative humidity.
1) Main peak including basic 0 peak.

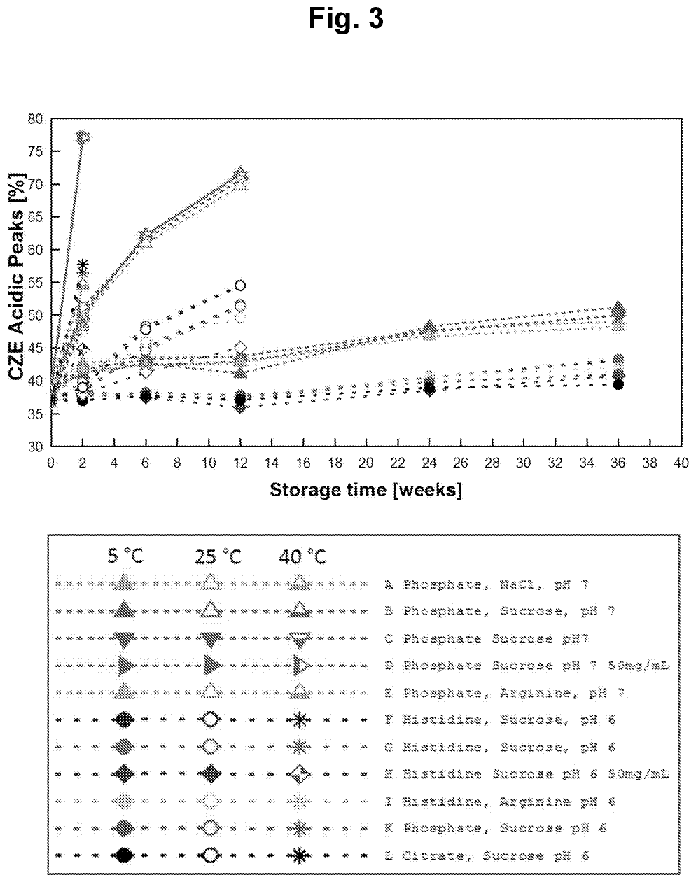

In contrast to the pH-dependent different development of basic peaks, acidic variants increase at both pH values (FIG. 3) over time, compared to the starting material. At pH 7.0, the observed increase in relative peak area of acidic variants is at all temperatures about twice the amount compared to pH 6.0. Again, at pH 6.0 only a very small increase in acidic variants is detectable at 2-8° C.

It can therefore be concluded that formulations at pH 6.0 show less absolute change in charge variant profiles over time and provide a more consistent product quality during storage compared to formulations at pH 7.0.

TABLE 8
Impact of formulation pH on CZE acidic peaks. Values in columns
A-K represent the sum of acidic variants’ % AUC in CZE diagram.

| | A | B | C | D | E | F | G | H | I | J | K |
|---|---|---|---|---|---|---|---|---|---|---|---|
| Initial | 39.8 | 39.1 | 38.5 | 38.9 | 37.6 | 37.4 | 37.7 | 37.2 | 36.6 | 36.8 | 37.0 |
| analysis | | | | | | | | | | | |
| (T = 0) | | | | | | | | | | | |
| 5° C./ | | | | | | | | | | | |
| ambient | | | | | | | | | | | |
| RH | | | | | | | | | | | |
| 2 weeks | 42.6 | 41.0 | 41.3 | 41.8 | 41.7 | 38.0 | 38.2 | 38.5 | 38.1 | 37.6 | 37.0 |
| 6 weeks | 43.7 | 42.7 | 42.4 | 43.2 | 42.3 | 37.5 | 37.9 | 37.4 | 37.8 | 38.2 | 37.7 |
| 12 weeks | 43.7 | 41.1 | 42.9 | 43.8 | 42.8 | 37.4 | 37.0 | 36.0 | 37.5 | 37.8 | 37.1 |
| 6 months | 47.8 | 48.3 | 47.4 | 47.6 | 46.8 | 40.5 | 40.5 | 38.5 | 40.7 | 39.8 | 38.9 |
| 9 months | 49.1 | 51.2 | 50.0 | 49.9 | 48.2 | 43.1 | 43.3 | 40.8 | 42.0 | 41.0 | 39.4 |
| 25° C./60% | | | | | | | | | | | |
| RH | | | | | | | | | | | |
| 2 weeks | 50.5 | 49.9 | 50.5 | 51.2 | 49.6 | 38.3 | 38.6 | 38.0 | 38.7 | 39.8 | 39.0 |
| 6 weeks | 62.0 | 62.3 | 62.2 | 61.6 | 60.9 | 45.1 | 44.6 | 41.3 | 45.9 | 48.3 | 47.8 |
| 12 weeks | 71.7 | 71.5 | 71.3 | 70.7 | 69.7 | 51.6 | 51.3 | 45.1 | 49.6 | 54.6 | 54.5 |
| 40° C./75% | | | | | | | | | | | |
| RH | | | | | | | | | | | |
| 2 weeks | 54.4 | 77.1 | 77.2 | 77.1 | 54.7 | 48.2 | 49.1 | 44.8 | 47.8 | 56.5 | 57.6 |

RH: relative humidity.
1) Main peak including basic 0 peak.

In a separate experiment for formulation K, charge heterogeneity was measured by CZE (using the same methods as for the other formulations) in different time points (Table 9).

TABLE 9
Impact of formulation pH on CZE main, basic and acidic
peaks for the formulation K.

| | Main variant | Sum of basic | Sum of acidic |
|---|---|---|---|
| | [%] | variants [%] | variants [%] |
| Initial analysis (T = 0) | 39.5 | 24.8 | 35.7 |
| 5° C./ambient RH | | | |
| 1.5 months | 37.1 | 25.7 | 37.1 |
| 3 months | 37.0 | 25.9 | 37.1 |
| 6 months | 34.7 | 25.7 | 39.6 |
| 9 months | 33.3 | 25.8 | 40.9 |
| 12 months | 33.5 | 25.4 | 41.1 |
| 15 months | 30.9 | 25.6 | 43.5 |
| 18 months | 29.7 | 25.3 | 45.1 |
| 24 months | 27.1 | 24.8 | 48.1 |
| 25° C./60% RH | | | |
| 1.5 months | 21.7 | 30.4 | 47.9 |
| 3 months | 15.3 | 27.8 | 56.9 |
| 6 months | 10.6 | 23.4 | 66.1 |
| 30° C./75% RH | | | |
| 1.5 months | 15.3 | 28.4 | 56.3 |
| 3 months | 10.2 | 24.1 | 65.7 |
| 6 months | 8.1 | 18.4 | 73.5 |

RH: relative humidity.

### 1.3 Purity by Size Exclusion Chromatography (SEC)

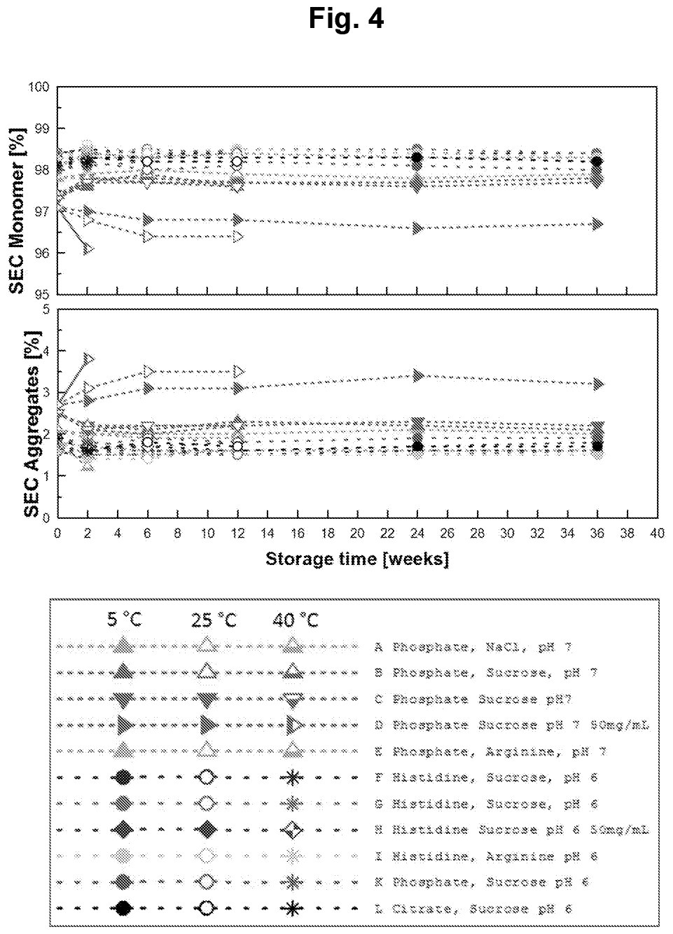

At all tested conditions, the level of aggregates is 1.5%-2% for formulations at pH 6.0 and 2%-2.5% for the 10 mg/mL formulations at pH 7.0 (FIG. 4, Tables 10-12). No relevant change in level of aggregates is observed. Fragments are present in quantities of approximately 0.1%. Monomer peak is >96% and also shows little changes over storage time. The 50 mg/mL formulation at pH 7.0 shows distinctly higher aggregate levels over the storage time, with up to ˜4% at stressed conditions.

TABLE 10
Purity of the screened formulations A-K as measured by SEC. The values
represent the % AUC corresponding to the monomer form of the antibody.

| | A | B | C | D | E | F | G | H | I | J | K |
|---|---|---|---|---|---|---|---|---|---|---|---|
| Initial | 97.8 | 97.4 | 97.3 | 97.1 | 98.1 | 98.4 | 98.4 | 98.3 | 98.3 | 98.0 | 98.1 |
| analysis | | | | | | | | | | | |
| (T = 0) | | | | | | | | | | | |
| 5° C./ | | | | | | | | | | | |
| ambient | | | | | | | | | | | |
| RH | | | | | | | | | | | |
| 2 weeks | 97.9 | 97.8 | 97.7 | 97.0 | 98.3 | 98.4 | 98.4 | 98.3 | 98.4 | 98.1 | 98.3 |
| 6 weeks | 98.0 | 97.8 | 97.7 | 96.8 | 98.3 | 98.3 | 98.4 | 98.3 | 98.4 | 98.2 | 98.3 |
| 12 weeks | 97.9 | 97.7 | 97.7 | 96.8 | 98.4 | 98.5 | 98.5 | 98.3 | 98.5 | 98.2 | 98.3 |
| 6 months | 97.8 | 97.7 | 97.6 | 96.6 | 98.3 | 98.4 | 98.5 | 98.3 | 98.4 | 98.1 | 98.3 |
| 9 months | 97.9 | 97.8 | 97.7 | 96.7 | 98.3 | 98.4 | 98.4 | 98.2 | 98.3 | 98.0 | 98.2 |
| 25° C./60% | | | | | | | | | | | |
| RH | | | | | | | | | | | |
| 2 weeks | 97.9 | 97.7 | 97.8 | 96.8 | 98.4 | 98.5 | 98.4 | 98.3 | 98.6 | 98.2 | 98.3 |
| 6 weeks | 98.0 | 97.9 | 97.7 | 96.4 | 98.3 | 98.4 | 98.5 | 98.2 | 98.4 | 98.0 | 98.2 |
| 12 weeks | 97.9 | 97.6 | 97.6 | 96.4 | 98.4 | 98.4 | 98.4 | 98.2 | 98.3 | 98.1 | 98.2 |
| 40^{o} C./75% | | | | | | | | | | | |
| RH | | | | | | | | | | | |
| 2 weeks | 97.8 | 97.6 | 97.7 | 96.1 | 98.4 | 98.5 | 98.4 | 98.2 | 98.5 | 98.1 | 98.2 |

RH: relative humidity.

TABLE 11
Purity of the screened formulations A-K as measured by SEC. The
values represent the sum of the antibody aggregate forms’ % AUC.

| | A | B | C | D | E | F | G | H | I | J | K |
|---|---|---|---|---|---|---|---|---|---|---|---|
| Initial | 2.1 | 2.5 | 2.5 | 2.7 | 1.8 | 1.6 | 1.6 | 1.6 | 1.6 | 1.9 | 1.9 |
| analysis | | | | | | | | | | | |
| (T = 0) | | | | | | | | | | | |
| 5° C./ | | | | | | | | | | | |
| ambient | | | | | | | | | | | |
| RH | | | | | | | | | | | |
| 2 weeks | 2.0 | 2.2 | 2.2 | 2.8 | 1.7 | 1.6 | 1.6 | 1.6 | 1.5 | 1.8 | 1.7 |
| 6 weeks | 2.0 | 2.1 | 2.2 | 3.1 | 1.6 | 1.6 | 1.5 | 1.6 | 1.5 | 1.7 | 1.7 |
| 12 weeks | 2.0 | 2.3 | 2.2 | 3.1 | 1.6 | 1.5 | 1.5 | 1.6 | 1.5 | 1.8 | 1.6 |
| 6 months | 2.1 | 2.2 | 2.3 | 3.4 | 1.6 | 1.6 | 1.5 | 1.7 | 1.5 | 1.9 | 1.7 |
| 9 months | 2.0 | 2.1 | 2.2 | 3.2 | 1.6 | 1.6 | 1.6 | 1.8 | 1.5 | 1.9 | 1.7 |
| 25° C./60% | | | | | | | | | | | |
| RH | | | | | | | | | | | |
| 2 weeks | 2.0 | 2.1 | 2.1 | 3.1 | 1.5 | 1.5 | 1.5 | 1.6 | 1.4 | 1.7 | 1.6 |
| 6 weeks | 1.9 | 2.0 | 2.2 | 3.5 | 1.5 | 1.5 | 1.5 | 1.7 | 1.4 | 1.9 | 1.8 |
| 12 weeks | 1.9 | 2.2 | 2.2 | 3.5 | 1.5 | 1.5 | 1.5 | 1.8 | 1.5 | 1.8 | 1.7 |
| 40° C./75% | | | | | | | | | | | |
| RH | | | | | | | | | | | |
| 2 weeks | 1.9 | 2.2 | 2.1 | 3.6 | 1.2 | 1.4 | 1.5 | 1.7 | 1.4 | 1.8 | 1.6 |

RH: relative humidity.

TABLE 12
Purity of the screened formulations A-K as measured by SEC. The
values represent the sum of the antibody fragments’ % AUC.

| | A | B | C | D | E | F | G | H | I | J | K |
|---|---|---|---|---|---|---|---|---|---|---|---|
| Initial | <0.10 | <0.10 | <0.10 | <0.10 | <0.10 | <0.10 | <0.10 | <0.10 | <0.10 | <0.10 | <0.10 |
| analysis | | | | | | | | | | | |
| (T = 0) | | | | | | | | | | | |
| 5° C./ | | | | | | | | | | | |
| ambient | | | | | | | | | | | |
| RH | | | | | | | | | | | |
| 2 weeks | <0.10 | <0.10 | <0.10 | <0.10 | <0.10 | <0.10 | <0.10 | <0.10 | <0.10 | <0.10 | <0.10 |
| 6 weeks | <0.10 | <0.10 | <0.10 | <0.10 | <0.10 | 0.1 | <0.10 | <0.10 | <0.10 | <0.10 | <0.10 |
| 12 weeks | <0.10 | <0.10 | <0.10 | <0.10 | <0.10 | <0.10 | <0.10 | <0.10 | <0.10 | <0.10 | <0.10 |
| 6 months | <0.10 | <0.10 | <0.10 | <0.10 | <0.10 | <0.10 | <0.10 | <0.10 | <0.10 | <0.10 | <0.10 |
| 9 months | <0.10 | <0.10 | <0.10 | <0.10 | <0.10 | <0.10 | <0.10 | <0.10 | <0.10 | <0.10 | <0.10 |
| 25° C./60% | | | | | | | | | | | |
| RH | | | | | | | | | | | |
| 2 weeks | 0.11 | 0.11 | <0.10 | <0.10 | <0.10 | <0.10 | <0.10 | <0.10 | <0.10 | <0.10 | <0.10 |
| 6 weeks | <0.10 | <0.10 | <0.10 | <0.10 | <0.10 | <0.10 | <0.10 | <0.10 | 0.11 | <0.10 | <0.10 |
| 12 weeks | <0.10 | <0.10 | <0.10 | <0.10 | 0.12 | <0.10 | <0.10 | <0.10 | 0.15 | <0.10 | <0.10 |
| 40° C ./75% | | | | | | | | | | | |
| RH | | | | | | | | | | | |
| 2 weeks | 0.16 | 0.13 | <0.10 | 0.18 | 0.21 | <0.10 | 0.13 | <0.10 | 0.10 | <0.10 | <0.10 |

RH: relative humidity.

In a separate experiment for formulation K, purity was measured by SEC (using the same methods as for the other formulations) in different time points (Table 13).

TABLE 13
Purity of the formulation K as measured by SEC.

| | Purity (monomer) | Sum of | Sum of |
|---|---|---|---|
| | [%] | aggregates [%] | fragments [%] |
| Initial analysis (T = 0) | 98.9 | 0.54 | <0.10 |
| 5° C./ambient RH | | | |
| 1.5 months | 98.9 | 0.55 | <0.10 |
| 3 months | 98.8 | 0.60 | <0.10 |
| 6 months | 99.0 | 0.62 | <0.10 |
| 9 months | 98.8 | 0.71 | <0.10 |
| 12 months | 98.7 | 0.75 | <0.10 |
| 15 months | 98.7 | 0.75 | <0.10 |
| 18 months | 98.8 | 0.78 | <0.10 |
| 24 months | 98.7 | 0.82 | <0.10 |
| 25° C./60% RH | | | |
| 1.5 months | 98.8 | 0.68 | <0.10 |
| 3 months | 98.8 | 0.75 | <0.10 |
| 6 months | 98.9 | 0.78 | <0.10 |
| 30° C./75% RH | | | |
| 1.5 months | 98.8 | 0.71 | 0.10 |
| 3 months | 98.8 | 0.79 | <0.10 |
| 6 months | 98.7 | 0.85 | 0.10 |

RH: relative humidity.

### Example 2: Assays for Measuring Bioactivity of SEG101

### 2.1 ELISA

In the ELISA, potency and identity of SEG101 samples are measured based on their ability to bind recombinant human P-selectin. ELISA plates are coated with recombinant human P-selectin, and graded amounts of SEG101 are added. Bound SEG101 is quantified using an anti-human IgG antibody coupled to horseradish peroxidase followed by the addition of a colorimetric substrate. Results of the colorimetric reaction are measured by light absorption. The potency of a SEG101 test sample is quantified by comparing its ability to bind P-selectin to that of a SEG101 reference standard. The samples and the standard are normalized on the basis of protein content. Relative potency is calculated using a parallel line assay according to the European Pharmacopoeia. The final result is expressed as relative potency (in percent) of a sample compared to the reference standard.

### 2.2 Cell-Based Assay—Inhibition of Adhesion of P-Selectin Expressing Raji Cells to PSGL-1

V-bottom microtiter plates are coated with recombinant human PSGL-1, and then graded amounts of SEG101 are added. Raji cells, recombinantly modified to present human P-selectin on their surface, are fluorescently labelled and added to the plate. After incubation, the plate is centrifuged and not adhered cells accumulate at the bottom of the V-shaped wells, where they are measured using a fluorescence reader. The potency of a SEG101 test sample is quantified by comparing its ability to inhibit the adhesion of P-selectin expressing Raji cells to PSGL-1 to that of a SEG101 reference standard. The samples and the standard are normalized on the basis of protein content. Relative potency is calculated using a parallel line assay according to the European Pharmacopoeia. The final result is expressed as relative potency (in percent) of a sample compared to the reference standard.

TABLE 14
Bioactivity of different charge variants of crizanlizumab.

| Peak comprising | P-selectin binding | Bioactivity cell- |
|---|---|---|
| Variant | affinity [%] | based assay [%] |
| D-Succi-crizanlizumab | 78 | 45 |
| isoD-Succi-crizanlizumab | 63 | 33 |
| Crizanlizumab | 90 | 102 |
| Hetero-iso-crizanlizumab | 105 | 125 |
| Homo-iso-crizanlizumab | 119 | 141 |

### Example 3: Purity by Size Exclusion Chromatography (SEC)

This test is based on Size Exclusion Chromatography (SEC) with UV detection. The variants of different size (e.g. lower and higher molecular weight variants and impurities) are separated by SEC under native conditions on a suitable column. The purity of the main peak as well as the amount of aggregates and fragments is determined as a percentage of the total area obtained for the sample in each chromatogram.

The HPLC-system to be used is a suitable high performance liquid chromatography system equipped with a pump, injection system and an online degasser, autosampler with cooling device, column heat and a UV detector capable of monitoring absorbance at 210 nm. The column is TSKgel G3000SWXL, 5 μm; 7.8 mm×300 mm, or Equivalent.

The sample solution is diluted with mobile phase (150 mM potassium phosphate solution at pH 6.5±0.1) to a final concentration of approximately 0.75 mg SEG101/mL. In order to make the Reference solution, the reference substance is diluted with mobile phase to a final concentration of approximately 0.75 mg SEG101/mL. Mobile phase is used as Blank. LOQ solution is made by diluting the reference substance with aprotinin solution to a final concentration of approximately 0.75 μg SEG101/mL, 2 mg aprotinin/mL.

Chromatographic conditions were adjusted as flow rate 0.4 mL/min, detection with UV 210 nm, column temperature 30° C. ±2° C., auto-sampler temperature approximately 5° C., run time 35 minutes, and injection volume 10 μL of the test and reference solutions, equivalent to approximately 7.5 μg of SEG101 in the reference solution. In the blank chromatogram no interfering peak should be detected in the blank with a signal height≥LOQ signal height, in the integrated range of the chromatogram of the test solution. Limit of quantitation (LOQ) is so that signal-to-noise ratio ≥10 of SEG101 peak. Reproducibility of the peak area should be so that PAₛₐₘₚₗₑ divided by PA_{ref} is at least 0.80 and maximum 1.20. PAₛₐₘₚₗₑ refers to the total peak areas for each individual sample injection in mAU×min. PA_{ref} refers to the total peak area for the reference injection before the sample block in mAU×min.

The Purity (% P of the main peak), the sum (%) of aggregates (eluting earlier than the main component) without the peak at relative retention time of 0.95 and the sum (%) of fragments (eluting later than the main component) are to be reported. The peak at a relative retention time of 0.95 (retention time relative to the retention time of the monomer/main peak) is to be reported individually.

### Example 4: Identity and Charge Heterogeneity by CZE

The separation principle of capillary zone electrophoresis (CZE) is based on the different electrophoretic mobility of proteins with different net charge to mass ratios in an electric field. At a pH below its pI each protein is positively charged and will migrate from anode to cathode. The migration velocity of a protein variant compared to the main peak is higher with increased net charge to mass ratio of the protein variant. For the sake of clarity the term “main peak” refers to crizanlizumab, even though under certain conditions the predominant form in the pharmaceutical composition is the iso-crizanlizumab form. Although a variety of reasons may result in different mobility, variants migrating faster than the main variant are generally named ‘basic variants’ and those migrating slower are named ‘acidic variants’. After detection by UV absorbance the charge variants are quantified by relative time corrected peak area determination.

The identity is determined by observing the main peak pattern in the co-mix or by comparing the electropherograms.

A capillary electrophoresis system with UV detector capable of detection at 214 nm can be used. Capillary is uncoated fused-silica capillary, internal diameter 50 μm.

The sample solution is diluted with sample buffer (5 mM phosphate pH 7.3±0.1) to a final concentration of approximately 3.0 mg SEG101/mL. The reference substance is diluted with sample buffer to a final concentration of approximately 3.0 mg SEG101/mL. The Sensitivity solution is made by diluting the reference substance with sample buffer to a final concentration of approximately 60 μg SEG101/mL. The Co-mix solution is made by mixing the test solution with the reference solution at the ratio 3:2 (v/v). Sample buffer is used as Blank.

Electrophoretic conditions are adjusted so that capillary length from inlet to detector is 40 cm, total capillary length of capillary is 50 cm, voltage is 20 kV, polarity is positive, capillary temperature is 25° C.±2° C., auto-sampler temperature is 15° C.±3° C., run time is 45 minutes, data rate is 8 Hz, detection is at 214 nm and aperture is 100×800 μm, and injection time is 0.5 psi for 8 s (4 psi×s).

No interfering peak should be detected in the blank with a signal height ≥LOQ signal height, in the integrated range of the electropherogram of the sample. Resolution should be R_{S}≥1.4, wherein the resolution R_{S} is calculated for the first and last injection of the reference solution to assess the capillary performance. In order to confirm the limit of quantitation (0.60%) the signal-to-noise ratio (S/N) for the SEG101 main peak in the LOQ solution (2.0% dilution) must be ≥10. Reproducibility of the peak area should be so that PAₛₐₘₚₗₑ divided by PA_{ref} is at least 0.80 and maximum 1.20. PAₛₐₘₚₗₑ refers to the total time corrected peak area for each individual injection of the test solution (mAU), and PA_{ref} refers to the total time corrected peak area for the first injection of the reference solution (mAU).

For purity, for each sample the relative time corrected peak areas of acidic variants (migration times >1.0 relative to the main peak), the relative time corrected peak areas of basic variants (migration times <1.0 relative to the main peak) and the main peak are to be reported. Charge variants <0.60% (LOQ) are not included in the calculation of the sum of acidic or basic variants.

For identity, the single peak observed for the main peak in the co-mix solution is required. Additionally, the peak pattern of the sample solution is compared to the peak pattern of the reference.

### Example 5: Preparation of Lyophilized Formulations for SEG101

Table 2 shows liquid formulations tested for their suitability for SEG101.

SEG101 was also formulated in 6 different lyophilized formulations, covering a range of different protein concentrations, lyoprotectant to protein molar ratio and the use of amorphous lyoprotectant alone (sucrose) or in combination with crystalline bulking agent (mannitol). The exact composition of the formulations is presented in Table 15.

TABLE 15
Composition of SEG101 pre-lyophilized formulations.

| Formulation | DP1 | DP2 | DP3 | DP4 | DP5 | DP6 |
|---|---|---|---|---|---|---|
| SEG101 [mg/ml] | 30 | 40 | 50 | 30 | 30 | 30 |
| Na Citrate buffer [mM] | 20 | 20 | 20 | 20 | 20 | 20 |
| PH | 6.0 | 6.0 | 6.0 | 6.0 | 6.0 | 6.0 |
| Sucrose [mg/mL] | 90 | 90 | 40 | 20 | 40 | 40 |
| Mannitol [mg/mL] | 0 | 0 | 0 | 40 | 80 | 60 |
| Polysorbate 80 [%] | 0.02 | 0.02 | 0.02 | 0.02 | 0.02 | 0.02 |
| Molar ratio (sucrose: SEG101) | 1245 | 933 | 332 | 277 | 553 | 553 |

### 5.1 Lyophilization Process Conditions

Due to difference in formulation composition, i.e. amorphous vs. partially crystalline formulations (Table 15), two distinct lyophilization processes were executed.

The first run summarized in Table 16 was selected for amorphous formulations DP1, DP2 and DP3. In the second run summarized in Table 17, formulations containing combination of sucrose and mannitol, i.e. DP4, DP5 and DP6 were lyophilized. Chamber pressure in both runs during PD phase was set to 0.133 mbar. Secondary drying phase (SD) was identical for both runs. Since the overall dry matter content is relatively high in candidate formulations (up to 443 mg in amorphous formulations and up to 557 mg in partially crystalline formulations), there is the risk of increased residual moisture level. Therefore, the chamber pressure during SD was reduced to 0.05 mbar and the set shelf temperature was 30° C. for 8 h.

TABLE 16
The first run parameters.

| | | Time | Total | Temp | Speed | set |
|---|---|---|---|---|---|---|
| Step | | [hh:mm] | Time | [° C.] | [° C./min] | pressure |
| 1 | Loading | 00:00 | 0 | 20 | 0.0 | 1000.00 |
| 2 | Precooling | 00:15 | 15 | 5 | −1.0 | 1000.00 |
| 3 | Precooling | 01:00 | 75 | 5 | 0.0 | 1000.00 |
| 4 | Freezing | 01:30 | 165 | −40 | −0.5 | 1000.00 |
| 5 | Freezing | 03:00 | 330 | −40 | 0.0 | 1000.00 |
| 6 | Primary drying | 00:50 | 396 | −15 | 0.5 | 0.133 |
| 7 | Primary drying | 40:00 | 2796 | −15 | 0.0 | 0.133 |
| 8 | Secondary drying | 01:30 | 2887 | 30 | 0.5 | 0.050 |
| 9 | Secondary drying | 08:00 | 3367 | 30 | 0.0 | 0.050 |
| | Total time [h]: | | 56 | | | |

TABLE 17
The second run parameters.

| | | | | | | set |
|---|---|---|---|---|---|---|
| | | Time | Total | Timetemp | Speed | pressure |
| Step | | [hh:mm] | [min] | [° C.] | [° C./min] | [mbar] |
| 1 | Loading | 00:00 | 0 | 20 | 0.0 | 1000 |
| 2 | Freezing | 00:15 | 15 | 5 | −1.0 | 1000 |
| 3 | Precooling | 01:00 | 75 | 5 | 0.0 | 1000 |
| 4 | Precooling | 01:30 | 165 | 40 | −0.5 | 1000 |
| 5 | Freezing | 03:00 | 345 | −40 | 0.0 | 1000 |
| 6 | Annealing | 00:40 | 385 | −20 | 0.5 | 1000 |
| 7 | Annealing | 02:00 | 505 | −20 | 0.0 | 1000 |
| 8 | Freezing | 00:40 | 545 | −40 | −0.5 | 1000 |
| 9 | Freezing | 02:00 | 665 | −40 | 0.0 | 1000 |
| 10 | Primary drying | 01:30 | 756 | 5 | 0.5 | 0.133 |
| 11 | Primary drying | 01:00 | 2256 | 5 | 0.0 | 0.133 |
| 12 | Secondary drying | 00:50 | 2307 | 30 | 0.5 | 0.050 |
| 13 | Secondary drying | 08:00 | 2787 | 30 | 0.0 | 0.050 |

| | |
|---|---|
| Total Time [h]: | 46 |

### 5.2 Stability

The objective of lyo stability study was to compare the physico-chemical stability of six lyophilised SEG101 formulations (Table 15). Formulations were exposed to intended (5±3° C.), accelerated (25±2° C., 60±5% r.h.; r.h.: relative humidity) and stress (40±2° C., 75±5% r.h.) storage conditions for up to 12 months and analysed.

Comparison of the results for pre-lyo solutions entering the lyophilization process with the results for reconstituted solutions right after the lyophilization cycle shows that the reconstitution and lyophilization cycle itself practically do not have an impact on physicochemical properties of the formulations.

### 5.3 Determination of Aggregates by SEC and Charge Variants by CZE

The lyophilized formulations were reconstituted in APW (additionally purified water) at different time points and subjected to analysis by SEC (Table 18) and CZE (Table 19).

TABLE 18
Stability data for SEC.

| | | | | Peak at the |
|---|---|---|---|---|
| | Sum of | | | relative |
| | aggregates | Monomer | Sum of | retention |
| | by SEC | purity by | fragments by | time of 0.95 |
| Sample name | [%] | SEC [%] | SEC [%] | by SEC [%] |
| DP1_pre-lyo | 0.72 | 98.46 | 0.44 | 0.38 |
| DP2_pre-lyo | 0.80 | 98.39 | 0.42 | 0.39 |
| DP3_pre-lyo | 0.96 | 98.22 | 0.45 | 0.37 |
| DP4_pre-lyo | 0.83 | 98.33 | 0.45 | 0.39 |
| DP5_pre-lyo | 0.65 | 98.53 | 0.44 | 0.39 |
| DP6_pre-lyo | 0.67 | 98.49 | 0.46 | 0.38 |
| DP1_40C 6m | 1.00 | 98.27 | 0.33 | 0.40 |
| DP2_40C_6m | 1.24 | 98.03 | 0.34 | 0.40 |
| DP3_40C_6m | 4.61 | 94.62 | 0.34 | 0.42 |
| DP4_40C_6m | 3.88 | 95.37 | 0.35 | 0.41 |
| DP5_40C_6m | 2.14 | 97.09 | 0.36 | 0.41 |
| DP6_40C_6m | 2.03 | 97.25 | 0.32 | 0.40 |
| DP1_5C_12m | 0.77 | 98.52 | 0.36 | 0.35 |
| DP2_5C_12m | 0.83 | 98.44 | 0.37 | 0.36 |
| DP3_5C_12m | 1.25 | 98.05 | 0.34 | 0.36 |
| DP4_5C_12m | 1.28 | 97.89 | 0.45 | 0.39 |
| DP5_5C_12m | 0.79 | 98.51 | 0.36 | 0.34 |
| DP6_5C_12m | 0.78 | 98.55 | 0.32 | 0.36 |
| DP1_25C_12m | 0.81 | 98.53 | 0.32 | 0.35 |
| DP2_25C_12m | 1.00 | 98.43 | 0.22 | 0.36 |
| DP3_25C_12m | 2.53 | 96.76 | 0.35 | 0.36 |
| DP4_25C_12m | 2.05 | 97.22 | 0.37 | 0.36 |
| DP5_25C_12m | 1.23 | 98.06 | 0.36 | 0.35 |
| DP6_25C_12m | 1.23 | 98.04 | 0.36 | 0.37 |

TABLE 19
Stability data for CZE.

| | | CZE | CZE | | | CZE | CZE |
|---|---|---|---|---|---|---|---|
| | | Sum | Sum | | | Sum | Sum |
| | CZE | of | of | | CZE | of | of |
| | Main | acidic | basic | | Main | acidic | basic |
| | variant | peaks | peaks | | variant | peaks | peaks |
| Sample name | [%] | [%] | [%] | Sample name | [%] | [%] | [%] |
| DP1_pre-lyo | 41.9 | 34.0 | 24.1 | DP1_5C_12m | 41.0 | 33.4 | 25.6 |
| DP2_pre-lyo | 41.8 | 34.1 | 24.1 | DP2_5C_12m | 41.0 | 33.5 | 25.5 |
| DP3_pre-lyo | 41.5 | 34.7 | 23.8 | DP3_5C_12m | 41.3 | 33.1 | 25.6 |
| DP4_pre-lyo | 41.3 | 34.6 | 24.2 | DP4_5C_12m | 40.7 | 33.4 | 26.0 |
| DP5_pre-lyo | 41.8 | 34.4 | 23.8 | DP5_5C_12m | 40.9 | 33.2 | 25.9 |
| DP6_pre-lyo | 41.8 | 34.5 | 23.7 | DP6_5C_12m | 40.9 | 33.5 | 25.7 |
| DP1_40C_6m | 37.7 | 37.7 | 24.6 | DP1_25C_12m | 39.8 | 34.3 | 25.9 |
| DP2_40C_6m | 38.1 | 37.1 | 24.8 | DP2_25C_12m | 40.3 | 33.3 | 26.4 |
| DP3_40C_6m | 34.3 | 38.7 | 27.0 | DP3_25C_12m | 38.6 | 33.8 | 27.7 |
| DP4_40C_6m | 34.5 | 38.5 | 27.0 | DP4_25C_12m | 38.5 | 34.2 | 27.3 |
| DP5_40C_6m | 36.4 | 37.2 | 26.4 | DP5_25C_12m | 39.1 | 34.3 | 26.6 |
| DP6_40C_6m | 36.4 | 37.4 | 26.2 | DP6_25C_12m | 40.0 | 33.4 | 26.6 |

### 5.4 Further Studies on DP2

### 5.4.1 Long Term Stability Testing (5° C.)

After 6 months storage of DP2 at 5° C., all results are within the requirements set for long term storage conditions. No trends were observed for all quality characteristics tested.

### 5.4.2 Accelerated Stability Testing (25° C./60 Percent RH)

After 6 months storage of DP2 at 25° C./60% RH, no trends were observed for all quality characteristics, except for the results presented in Table 20. The results for all other methods follow the trend expected for accelerated conditions. All results are within the requirements set for long-term storage conditions.

TABLE 20
Charge variants in DP2 stored at 25° C. at
different time points as measured by CZE.

| | Main | Sum of basic | Sum of acidic |
|---|---|---|---|
| | variant [%] | variants [%] | variants [%] |
| Initial analysis (t = 0) | 39.6 | 24.7 | 36.0 |
| 1.5 months | 38.9 | 25.2 | 36.0 |
| 3 months | 39.4 | 26.1 | 34.6 |
| 6 months | 37.3 | 27.0 | 35.7 |

### 5.4.3 Stress Stability Testing (40 Degree Celsius/75 Percent RH)

After 6 months storage of DP2 at 40° C./75% RH, no trends were observed for all quality characteristics, except for the results presented in Table 21.

The results for all other methods follow the trend expected for stress conditions. All results are within the requirements set for long term storage conditions.

TABLE 21
Changes observed during stress testing for DP2.

| | Charge heterogeneity by CZE | | | Purity by size exclusion chromatography (SEC) | | | |
|---|---|---|---|---|---|---|---|
| | | Sum of | Sum of | | | | Peak at |
| | Main | basic | acidic | Purity | Sum of | Sum of | rRT |
| | variant | variants | variants | (monomer) | aggregates | fragments | 0.95 |
| | [%] | [%] | [%] | [%] | [%] | [%] | [%] |
| Initial | 39.6 | 24.7 | 36.0 | 98.7 | 0.91 | <0.10 | 0.35 |
| analysis (t = 0) | | | | | | | |
| 1.5 months | 37.6 | 25.9 | 36.6 | 98.5 | 1.1 | <0.10 | 0.37 |
| 3 months | 37.7 | 26.7 | 35.6 | 98.4 | 1.2 | <0.10 | 0.36 |
| 6 month | 36.0 | 27.8 | 36.1 | 98.3 | 1.3 | <0.10 | 0.36 |

rRT: Relative retention time.

### 5.5 Results for Other Analytical Methods

All the reconstituted formulations were practically free of visible particles and colorless at all the pull points. UV content and pH were unchanged (within the method variability or expected vial to vial variability) at all stress conditions. The biological activity of formulations DP1, DP2 and DP3 as determined with ELISA and cell-based assay was unchanged (within the expected method variability) after 6 months at 40° C., and for DP1 and DP2 after 12 months at 5° C. and 25° C. (Table 22).

TABLE 22
Lyo stability data for ELISA and Bioassay.

| | ELISA-relative | Cell-based assay-relative |
|---|---|---|
| Sample name | potency [%] | potency [%] |
| DP1_40C_3m | 94 | 85 |
| DP2_40C_3m | 88 | 89 |
| DP3_40C_3m | 90 | 101 |
| DP1_40C_6m | 91 | 97 |
| DP2_40C_6m | 91 | 97 |
| DP3_40C_6m | 92 | 100 |
| DP1_5C_12m | 91 | 101 |
| DP2_5C_12m | 100 | 99 |
| DP1_25C_12m | 85 | 102 |
| DP2_25C_12m | 96 | 100 |

### Example 6: Measuring P-Selectin Inhibition by Crizanlizumab and any Variants Thereof in Human Samples (PD) Using Surface Plasmon Resonance Analysis (SPR)

An SPR-based method was used to measure ex vivo % P-selectin inhibition of crizanlizumab in human serum samples from human donors treated with crizanlizumab. This PD assay measured the ability of crizanlizumab and it variants in serum to block P-selectin (antigen; ligand) binding to PSGL-1 (ligand receptor). Blockage by crizanlizumab and any variants thereof was measured as % inhibition of spiked P-selectin fused to immunoglobulin (Psel-Ig) binding to glycosulfopeptide 6 (GSP-6), a peptide analogue mimicking the PSGL-1 binding domain. For example, streptavidin was immobilized on 2 channels (FC-2 and FC-3) of the used biosensor chip using standard amine coupling followed by injection of biotinylated GSP-6 to immobilize the peptide for analysis. A reference channel (FC-1) was prepared by biotin-blocking streptavidin. The pre-treatment samples established the maximum binding for each subject and this was used as the basis for calculating % inhibition using the post-dose serum samples from the subject at various time points. Quantified crizanlizumab and any variants thereof in a series of dilutions were used to generate standard inhibitition curve, showing an IC₅₀ value of 5.2 μg/mL for crizanlizumab under all conditions.

Serum samples from patients with SCD mixed with blocking buffer without Psel-Ig and with Psel-Ig served as negative control and positive control samples, respectively.

The assay shows that crizanlizumab and any variants thereof in serum collected from patients does actually bind to the target. Crizanlizumab and any variants thereof in patient serum blocked 98% of spiked P-selectin (Psel-Ig) binding to glycosulfopeptide 6 (GSP-6).

1. A pharmaceutical composition comprising crizanlizumab that has light chain and heavy chain amino acid sequences in SEQ ID NO: 10 and SEQ ID NO: 9 respectively, and a variant of crizanlizumab (iso-crizanlizumab), in which amino acid aspartic acid at position 32 of SEQ ID NO: 10 is changed to iso-aspartic acid.

2. The pharmaceutical composition of claim 1 further comprising succinimide of crizanlizumab at position 32 of SEQ ID NO: 10.

3. The pharmaceutical composition of claim 1 or 2, wherein the iso-crizanlizumab consists of homo-iso-crizanlizumab and hetero-iso-crizanlizumab.

4. The pharmaceutical composition of any one of the claims 1 to 3 comprising at least 20% crizanlizumab of the total charge variants in the pharmaceutical composition.

5. The pharmaceutical composition of any one of the claims 1 to 4 comprising at most 50% crizanlizumab of the total charge variants in the pharmaceutical composition.

6. The pharmaceutical composition of any one of the claims 1 to 5 comprising from about 20% to about 50% crizanlizumab of the total charge variants in the pharmaceutical composition.

7. The pharmaceutical composition of any one of the preceding claims further comprising a buffer system, wherein the composition has a pH from about 5.5 to about 7.5.

8. The pharmaceutical composition of claim 7, wherein the pH is about 5.5 to about 7, preferably about 5.5 to about 6.5, preferably about 5.7 to about 6.3.

9. The pharmaceutical composition of claim 7 or 8, wherein the pH is about 5.9 to about 6.1.

10. The pharmaceutical composition of any one of claims the 7-9, wherein the buffer system is citrate buffer.

11. The pharmaceutical composition of any one of the claims 7-9, wherein the buffer system is phosphate buffer.

12. The pharmaceutical composition of any one of the preceding claims further comprising a stabilizer.

13. The pharmaceutical composition of claim 12, wherein the stabilizer is sucrose.

14. The pharmaceutical composition of claim 13, wherein sucrose is in a concentration of 50 mM to 350 mM, preferably 100 mM to 300 mM.

15. The pharmaceutical composition of any one of the preceding claims further comprising an isotonizing agent.

16. The pharmaceutical composition of claim 15, wherein the isotonizing agent is sodium chloride.

17. The pharmaceutical composition of claim 16, wherein sodium chloride is in a concentration of 50 mM to 300 mM.

18. The pharmaceutical composition of any one of the preceding claims further comprising a surfactant.

19. The pharmaceutical composition of claim 18, wherein the surfactant is a non-ionic surfactant.

20. The pharmaceutical composition of claim 18 or 19, wherein the surfactant is polysorbate, preferably polysorbate 80.

21. The pharmaceutical composition of any of the claims 18 to 20, wherein the surfactant, preferably polysorbate, is in a concentration of 0.01% w/v (0.1 mg/mL) to 0.1% w/v (1 mg/mL).

22. The pharmaceutical composition of any one of the preceding claims, wherein ANTIBODY is in a concentration from 5 mg/ml to 50 mg/ml.

23. A pharmaceutical composition comprising antibody in a concentration from about 5 to about 50 mg/ml, and a buffer system, wherein the composition has a pH from about 5.5 to about 7.5.

24. The pharmaceutical composition according to claim 23, wherein the pH is about 5.7 to about 6.3.

25. The pharmaceutical composition according to claim 23 or 24, wherein the buffer system is citrate (e.g. sodium citrate) and/or phosphate (e.g. potassium phosphate).

26. The pharmaceutical composition according to any one of the claims 23-25 further comprising a stabilizer.

27. The pharmaceutical composition according to claim 26, wherein the stabilizer is sucrose.

28. The pharmaceutical composition according to claim 26 or 27, wherein the stabilizer is present in a concentration of about 50 mM to about 300 mM.

29. The pharmaceutical composition according to any one of the claims 23-28 further comprising a non-ionic surfactant.

30. The pharmaceutical composition according to claim 29, wherein the surfactant is polysorbate 80.

31. The pharmaceutical composition according to claim 29 or 30, wherein the surfactant is present in a concentration of about 0.01% w/v (0.1 mg/ml) to 0.1% w/v (1 mg/ml).

32. The pharmaceutical composition according to any one of the claims 23-31 further comprising an isotonizing agent.

33. The pharmaceutical composition according to claim 32, wherein the isotonizing agent is NaCl.

34. The pharmaceutical composition according to claim 32 or 33, wherein the isotonizing agent is present in a concentration of about 50 mM to 300 mM.

35. A pharmaceutical composition comprising antibody (crizanlizumab and any variants thereof) in a concentration from about 5 to about 50 mg/ml, sucrose in a concentration of about 50 mM to about 350 mM, and a buffer system, wherein the composition has a pH from about 5.5 to about 7.5, and wherein the buffer system is citrate buffer and/or phosphate buffer.

36. The pharmaceutical composition according to claim 35, wherein the pH of the pharmaceutical composition is about 5.7 to about 6.3, e.g. about 6.0.

37. The pharmaceutical composition according to any one of the preceding claims in a lyophilized form.

38. A lyophilized formulation obtainable by lyophilizing an aqueous formulation, wherein the lyophilized formulation comprises:
a) antibody (crizanlizumab and any variants thereof);
b) a lyoprotectant; and
c) a buffer system.

39. The lyophilized formulation of claim 38 further comprising a surfactant.

40. The lyophilized formulation of claim 39, wherein said surfactant is polysorbate 80.

41. The lyophilized formulation of any one of the claims 38-40, wherein antibody is present in the aqueous formulation in a concentration of about 10 mg/mL to 100 mg/mL.

42. The lyophilized formulation of any one of the claims 38-41, wherein the buffer system is citrate, e.g. sodium citrate.

43. The lyophilized formulation of any one of the claims 38-42 further comprising sucrose and/or mannitol as lyoprotectant.

44. The lyophilized formulation of any of claims 38-43, wherein said aqueous formulation comprises sucrose in a concentration of about 10 mg/mL to 100 mg/mL.

45. The lyophilized formulation of any of claims 43-44, wherein the molar ratio of sucrose to antibody is from about 200 to 1500.

46. The lyophilized formulation of any of claims 38-45 comprising
a) about 25-40 w/w %, preferably about 28 to 32 w/w % , of antibody; and
b) about 55-75 w/w 00 preferably about 65 to 71 w/w % of sucrose,
based on the total weight of the lyophilized formulation.

47. A liquid pharmaceutical composition obtained by reconstituting the lyophilized formulation of any one of the claims 37 to 46.

48. The pharmaceutical composition according to any one of the preceding claims, wherein the IC50 determined by using the pharmaceutical composition is in the range of about 4.6-6.2 μg/ml.

49. The pharmaceutical composition according to claim 48, wherein the IC50 is determined in vitro.

50. A method of treating sickle cell disease, especially preventing Vascular Occlusion Crises (VOC) in a subject in need thereof comprising administering a therapeutically effective dose of antibody (crizanlizumab and any variants thereof) comprised in the pharmaceutical composition according to any one of the claims 1-47 to subject, wherein therapeutically effective dose is 5 mg or 7.5 mg per kilogram of the body weight of subject.

51. The method according to claim 50, wherein the first 2 doses are administered 2 weeks apart, followed by administration of the same dose every four weeks.

52. The method of claim 50 or 51, wherein the pharmaceutical composition is administered to a subject by intravenous route.

53. The method of any one of the claims 50 to 52, wherein therapeutically effective dose is 5 mg per kilogram of the weight of subject, wherein the first 2 doses are administered 2 weeks apart, followed by administration of the same dose every four weeks, and wherein one or more or all of the following PK parameters are met:
a) a tₘₐₓ in a range for from 0.4 to 10 hours (h), preferably from 0.55 h to 6.25 h, with a preferred Median of 1.5 to 2.5 h, preferably of 1.92 h, after administration of said pharmaceutical composition;
b) a Cₘₐₓ in the range of, after first dose, 116±91.3 μg/mL; or preferably at steady state at 50 to 200 μg/mL, preferably at 124±31.6 μg/mL;
c) an apparent t_{1/2} in the range of 100 h to 300 h, preferably in the range of 150 h to 210 h, e.g. at about 183 h (7.6 days);
d) an AUC_{tau, ss} in the range of 10 000 to 30 000 μg×h/mL, preferably at week 15 at 20400 μg×h/mL, preferably with a Coefficient of Variance of 23.5%;
e) a mean clearance at steady state week 15, preferably in a patient with SCD and a body weight of 70 kg, in the range of 10 to 30 mL/h, preferably 15 to 20 mL/h, e.g. at about 17.2 mL/h;
f) a PK trough concentration obtained every 4 weeks in the steady state, especially from week 7 to week 27, range from about 3.78 μg/mL to 9.8 μg/mL.

54. The method of any one of the claims 50 to 52, wherein crizanlizumab and any variants thereof in serum achieves at least 70%, at least 80%, at least 90%, at least 95% inhibition of binding of P-Selectin with PSGL-1.
